(19) United States
(12) Patent Application Publication
(10) Pub. No.: US 2020/0095557 A1
(43) Pub. Date: Mar. 26, 2020

(54) CELL SPHEROIDS CONTAINING CAPILLARY STRUCTURES AND METHODS OF USING SAME

(71) Applicant: Indiana University Research and Technology Corporation, Indianapolis, IN (US)

(72) Inventors: Nicanor I. Moldovan, Fishers, IN (US); Mervin C. Yoder, Indianapolis, IN (US); Nutan Prasain, Boston, MA (US)

(73) Assignee: Indiana University Research and Technology Corporation, Indianapolis, IN (US)

(21) Appl. No.: 16/580,954

(22) Filed: Sep. 24, 2019

Related U.S. Application Data
(60) Provisional application No. 62/735,790, filed on Sep. 24, 2018.

(51) Int. Cl. C12N 5/071; C12N 5/00; C12N 5/077

(52) U.S. Cl. CPC C12N 5/0697; C12N 5/0692; C12N 5/0062; C12N 2513/00; C12N 2506/45; C12N 2502/1347; C12N 2502/28; C12N 5/0661

(57) ABSTRACT

Co-culture of isolated human induced pluripotent stem cell (iPSC)-derived endothelial and smooth-muscle progenitor cells results in the formation of cellular spheroids having capillary-like structures (referred to herein as ‘capillary fragments’) at their core. Bioprinting of these spheroids into scaffold-free tissue constructs facilitates the development of microvasculature within the engineered tissue. Methods of using these bioprinted engineered tissues for cell therapy are also disclosed.

### CROSS-REFERENCE TO RELATED APPLICATIONS

This application claims the benefit of U.S. Provisional Application No. 62/735,790, filed Sep. 24, 2018, the entire disclosure of which is hereby incorporated by reference in its entirety.

### FIELD OF THE DISCLOSURE

The present disclosure pertains generally to the engineering of vascularized tissue constructs and their use for the replacement of tissues affected by disease or injury.

### SEQUENCE LISTING

The instant application contains a Sequence Listing which has been submitted via EFS-Web in computer readable form, and which is hereby incorporated by reference in its entirety. The ASCII copy, created on Sep. 24, 2018, is named IURTC-2017-085-01-US_SEQ_LIST_ST25.txt, and is 21,268 bytes in size.

### BACKGROUND

Tissue engineering is an emerging therapeutic option for the replacement of tissues affected by disease or injury. To survive, a transplanted tissue requires a constant supply of oxygenated blood and nutrients. Currently, tissue constructs are generated on artificial scaffolds having microvascular channels that act as capillaries. However, suboptimal blood perfusion through the transplanted tissue may nevertheless compromise its growth and/or long term viability. There is therefore an ongoing need in the art for improved tissue engineering methods that generate a tissue construct having a functional vasculature.

### SUMMARY

3D cell culture results in the formation of cell clusters called spheroids that more closely resemble cells in a live tissue than cells grown in a monolayer. Disclosed herein are cellular spheroids having an internal organization that facilitates the development of microvasculature within the spheroid itself, and methods of using the spheroids for cell therapy or for generating microvasculature in tissue engineering constructs. In various aspects, the cell spheroids disclosed herein are assembled from human induced pluripotent stem cell (iPSC)-derived endothelial progenitors and smooth-muscle forming cells that are able to organize into capillary-like structures (referred to herein as ‘capillary fragments’) at the core of each spheroid.

In a first aspect, the present disclosure provides a population of spheroids, wherein each spheroid comprises endothelial progenitor cells (EPCs)

In certain embodiments of the first aspect, the present disclosure provides a population of spheroids, wherein each spheroid comprises smooth muscle forming cells (SMFCs).

In certain embodiments of the first aspect, the present disclosure provides a population of spheroids, wherein each spheroid comprises endothelial progenitor cells (EPCs) and smooth muscle forming cells (SMFCs).

In certain embodiments of the first aspect, the endothelial progenitor cells (EPCs) are derived from a pluripotent stem cell population, peripheral blood or cord blood, or a combination of at least two thereof.

In certain embodiments of the first aspect, the endothelial progenitor cells (EPCs) are derived from CD31+NRP1+ cells isolated from a differentiated pluripotent stem cell population.

In certain embodiments of the first aspect, the SMFCs are derived from a pluripotent stem cell population.

In certain embodiments of the first aspect, the smooth muscle forming cells are derived from CD31+NRP1− cells isolated from a differentiated pluripotent stem cell population.

In certain embodiments of the first aspect, the population of pluripotent stem cells is clonal.

In certain embodiments of the first aspect, the pluripotent stem cells are induced pluripotent stem cells (iPSCs).

In certain embodiments of the first aspect, the EPCs comprise endothelial colony forming cells (ECFCs).

In certain embodiments of the first aspect, each spheroid comprises about 0.5% to about 20% EPCs and about 80% to about 99.5% SMFCs or about 20% EPCs and about 80% SMFCs.

In certain embodiments of the first aspect, the EPCs sort to the center of each spheroid.

In certain embodiments of the first aspect, the spheroids are substantially spherical having an average diameter of between about 200 microns and about 600 microns.

In certain embodiments of the first aspect, the average diameter of each spheroid is about 500 microns in diameter.

In certain embodiments of the first aspect, one or more capillary fragments form at the center of each spheroid.

In a second aspect, the present disclosure provides an engineered tissue comprising one or more spheroids of the any one of the preceding populations of spheroids.

In certain embodiments of the second aspect, the engineered tissue is bioprinted.

In a third aspect, the present disclosure provides a method of producing a vascularized tissue construct, comprising co-culturing endothelial colony progenitor cells (EPCs) and smooth muscle forming cells (SMFCs) to form a population of spheroids; and bioprinting the spheroids into a tissue construct.

In certain embodiments of the third aspect, the endothelial progenitor cells (EPCs) are derived from a pluripotent stem cell population, peripheral blood or cord blood, or a combination of at least two thereof.

In certain embodiments of the third aspect, the endothelial progenitor cells (EPCs) are derived from CD31+NRP1+ cells isolated from a differentiated pluripotent stem cell population.

In certain embodiments of the third aspect, the SMFCs are derived from a pluripotent stem cell population.

In certain embodiments of the third aspect, the smooth muscle forming cells (SMFCs) are derived from CD31+NRP1− cells isolated from a differentiated pluripotent stem cell population.

In certain embodiments of the third aspect, the population of pluripotent stem cells is clonal.

In certain embodiments of the third aspect, the pluripotent stem cells are induced pluripotent stem cells (iPSCs).

In certain embodiments of the third aspect, the EPCs comprise endothelial colony forming cells (ECFCs).

In certain embodiments of the third aspect, each spheroid comprises about 0.5% to about 20% EPCs and about 80% to about 99.5% SMFCs or about 20% EPCs and about 80% SMFCs.

In certain embodiments of the third aspect, the EPCs sort to the center of each spheroid.

In certain embodiments of the third aspect, the spheroids are substantially spherical having an average diameter of between about 200 microns and about 600 microns.

In certain embodiments of the third aspect, the average diameter of each spheroid is about 500 microns in diameter.

In certain embodiments of the third aspect, one or more capillary fragments form at the center of each spheroid.

In certain embodiments of the third aspect, the endothelial colony progenitor cells (EPCs) and smooth muscle forming cells (SMFCs) are co-cultured for 24 hours or less.

In certain embodiments of the third aspect, the endothelial colony progenitor cells (EPCs) and smooth muscle forming cells (SMFCs) are co-cultured for about 18 to 48 hours.

In certain embodiments of the third aspect, the endothelial colony progenitor cells (EPCs) and smooth muscle forming cells (SMFCs) are co-cultured for about 18 to 48 hours.

In certain embodiments of the third aspect, the endothelial colony progenitor cells (EPCs) and smooth muscle precursor cell (SMFCs) are co-cultured on an ultra-low attachment plate.

In certain embodiments of the third aspect, the endothelial colony progenitor cells (EPCs) and smooth muscle precursor cell (SMFCs) are co-cultured in an endothelial cell-supporting medium.

In certain embodiments of the third aspect, the tissue construct is scaffold-free.

In certain embodiments of the third aspect, the bioprinting comprises skewering the spheroids onto microneedles in a desired configuration.

In certain embodiments of the third aspect, the bioprinting comprises Kenzan bioprinting.

In certain embodiments of the third aspect, cells of the spheroids translocate during or after bioprinting to form a plurality of microvascular structures within the tissue construct.

In certain embodiments of the third aspect, a spheroid within the bioprinted tissue construct contacts at least one other spheroid.

In a fourth aspect, the present disclosure provides a method of seeding microvascular networks within a bioprinted tissue construct, comprising bioprinting a tissue construct with a population of tissue-specific spheroids and any one of the preceding populations of spheroids.

In certain embodiments of the fourth aspect, any one of the preceding populations of spheroids are produced by co-culturing endothelial colony progenitor cells (EPCs) and smooth muscle forming cells (SMFCs) for 24 hours or less

In certain embodiments of the fourth aspect, any one of the preceding populations of spheroids are produced by co-culturing endothelial colony progenitor cells (EPCs) and smooth muscle forming cells (SMFCs) for about 18 to 48 hours.

In certain embodiments of the fourth aspect, any one of the preceding populations of spheroids are produced by co-culturing endothelial colony progenitor cells (EPCs) and smooth muscle forming cells (SMFCs) on an ultra-low attachment plate.

In certain embodiments of the fourth aspect, any one of the preceding populations of spheroids are produced by co-culturing endothelial colony progenitor cells (EPCs) and smooth muscle forming cells (SMFCs) in an endothelial cell-supporting medium.

In certain embodiments of the fourth aspect, the tissue construct is scaffold-free.

In certain embodiments of the fourth aspect, the bioprinting comprises skewering spheroids from the populations of spheroids onto microneedles in a desired configuration.

In certain embodiments of the fourth aspect, the bioprinting comprises Kenzan bioprinting.

In certain embodiments of the fourth aspect, cells of the spheroids translocate during or after bioprinting to form a plurality of microvascular structures within the tissue construct.

In certain embodiments of the fourth aspect, a spheroid within the bioprinted tissue construct contacts at least one other spheroid.

In certain embodiments of the fourth aspect, the method further comprises combining the populations of spheroids with adult capillary fragments embedded in mesenchymal stem cells.

In certain embodiments of the fourth aspect, spheroids within the populations of spheroids comprises endothelial colony progenitor cells and smooth muscle forming cells are interspersed with the one or more tissue-specific spheroids at regular intervals.

In certain embodiments of the fourth aspect, the bioprinted tissue construct is selected from the group consisting of a cardiac patch, a vascular tube, a trachea, and a urethra.

### BRIEF DESCRIPTION OF THE DRAWINGS

The patent or application file contains at least one drawing executed in color.

FIG. 1A shows an embodiment of labeled iPSC-derived ‘endothelial colony forming cells’ settling as diffuse colonies (top) and forming irregular spheroids in U-shaped low-adhesive plates (bottom). FIG. 1B shows an embodiment of NRP1⁻CD31+-derived cells forming rounder colonies (top), then aggregating into well-defined spheroids (bottom). FIG. 1C shows an embodiment of a mixed 1:5 population of iPSC-derived ECFC and ‘smooth-muscle forming cells’ (SMFC) behaving more similarly to their SMFC-only counterparts. FIG. 1D illustrates an embodiment of a comparison between atypical cell spheroids observed after a four-day incubation and round cell spheroids.

FIG. 2 shows a representative time course of spheroid size as determined by fluorescence imaging.

FIG. 3 shows representative time-lapse snapshots of the formation of a mixed spheroid containing iPSC-derived ECFCs and SMFCs. A relatively stable spheroid size formed after about ˜18 hours, but the distribution of cell proportions on the surface changed from predominantly ECFCs (red) at 9 hours, to mostly SMFCs (green) at 48 hours. Last frame shows representative staining for caspase 3/7 activity in a 52 hours-old spheroid.

FIG. 4A shows a representative time course of biomarker fluorescence in spheroids made from ECFCs only or from 1:5 ECFCs/SMFCs spheroids. FIG. 4B shows a representative time course of biomarker fluorescence in spheroids prepared from NRP1⁻CD31⁺-derived cells and in ECFCs/SMFCs mixed spheroids. FIG. 4C shows the size distribution of spheroids depicted in FIG. 4A.

FIGS. 5A-5F show a representative cell distribution in early vs. late spheroids. FIG. 5A shows an embodiment of phase contrast imaging of a mixed one-day old spheroid. FIG. 5B shows an embodiment of radial positioning of an iPSC-ECFC cell cord within this early spheroid (arrow). FIG. 5C shows corresponding SMFCs within the same spheroid. FIGS. 5D-5F show an embodiment of successive confocal sections through a one-week old spheroid: note the extensive ‘cell sorting’ (layered separation), and the organization of the ECFCs in a fashion suggestive of cell cord formation, with possible sprouting viewed longitudinally (in FIG. 5D, arrowheads), or transversally (in FIG. 5E, arrowheads).

FIGS. 6A-6F show an embodiment of the characterization of a tissue construct from iPSC-derived SMFCs prepared on the Regenova™ bioprinter (operating with the Kenzan micro-needle method). FIG. 6A shows a representative micro-photograph of the tissue construct after one week of post-printing maturation. FIG. 6B shows an embodiment of whole-mount fluorescence microscopy, showing the eGFP-expressing cells (arrows, in the inserts), embedded in abundant, newly-secreted extracellular matrix (ECM) (pale-green, auto-fluorescence). FIG. 6C shows an embodiment of confocal microscopy of the tissue construct, identifying the eGFP-SMFCs (arrows). FIG. 6D shows the same section as FIG. 6C, with immunostaining for collagen type IV (red). FIG. 6E shows that CD31⁺NRP1⁻ cells differentiate into alpha-smooth muscle actin positive (yellow) and negative (fibroblastic) cells, the former being more concentrated on the surfaces of the construct. FIG. 6F shows a higher magnification of FIG. 6E.

FIGS. 7A-7F show an embodiment of the fusion and internal organization of iPSC-derived spheroids. FIG. 7A shows an embodiment of the in-well fusion of eight (FIG. 7A, 7B) and 16 (FIG. 7C, 7D) mixed 1:10 ECFCs/SMFCs, 24 hours-old spheroids. FIGS. 7E and 7F show an embodiment of Kenzan assembling of mixed spheroids. FIG. 7E shows an embodiment of ECFCs (red) with extended, cord-like circular organization of ECFCs in these bioprinted spheroids immediately after removal from the micro-needle array. FIG. 7F shows the corresponding SMFCs to FIG. 7E (green).

FIGS. 8A-8D show an bioprinted iPSC-derived construct. FIG. 8A shows a culture well from where a spheroid was taken. FIG. 8B shows mixed spheroids prior to bioprinting. FIG. 8C shows a lateral view of the microneedle array (Kenzan) with attached spheroids. FIG. 8D shows a top view of the same bioprinted iPSC cells-derived tube; the dark spots in the image represent the micro-needles (Kenzan).

FIG. 9 depicts an embodiment of the disclosed method of using tissue-specific spheroids and pre-formed capillary-containing spheroids to seed microvascular networks within a bioprinted tissue construct.

FIG. 10 depicts another embodiment of the disclosed method of using pre-formed capillary-containing spheroids to seed microvascular networks within a bioprinted tissue construct.

### DETAILED DESCRIPTION

While the disclosed subject matter is amenable to various modifications and alternative forms, specific embodiments are described herein in detail. The intention, however, is not to limit the disclosure to the particular embodiments described. On the contrary, the disclosure is intended to cover all modifications, equivalents, and alternatives falling within the scope of the disclosure as defined by the appended claims.

Similarly, although illustrative methods may be described herein, the description of the methods should not be interpreted as implying any requirement of, or particular order among or between, the various steps disclosed herein. However, certain embodiments may require certain steps and/or certain orders between certain steps, as may be explicitly described herein and/or as may be understood from the nature of the steps themselves (e.g., the performance of some steps may depend on the outcome of a previous step).

### Definitions

Unless explained otherwise, all technical and scientific terms used herein have the same meaning as commonly understood to one of ordinary skill in the art to which this disclosure belongs.

As used herein, the singular forms “a,” “an,” and “the,” are intended to include the plural forms as well, unless the context clearly indicates otherwise.

A “set,” “subset,” or “group” of items (e.g., inputs, algorithms, data values, etc.) may include one or more items, and, similarly, a subset or subgroup of items may include one or more items. A “plurality” means more than one.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%.

A “subject” is a vertebrate, preferably a mammal, more preferably a primate and still more preferably a human. Mammals include, but are not limited to, primates, humans, farm animals, zoo animals, rodents, sport animals, and pets.

By a “population of cells” or “cell population” is meant a collection of at least ten cells. Preferably, the population consists of at least twenty cells, more preferably at least one hundred cells, and most preferably at least one thousand, or one million or billions of cells.

By a “population of spheroids” is meant a collection of at least ten spheroids. Preferably, the population consists of at least twenty spheroids, more preferably at least one hundred spheroids, and most preferably at least one thousand spheroids, one million spheroids or billions of spheroids.

The terms “biofabrication,” “bioprinting”, and “bioassembling” refer to the utilization of 3D printing or 3D printing-like techniques to combine cells, other biomaterials, and/or growth factors to generate engineered tissues or biomedical parts which imitate natural tissue characteristics. Bioprinting generally utilizes a layer-by-layer method of depositing the materials using, for example, robotics, an inkjet printer, a laser-assisted printer, and/or an extrusion printer.

“Disease” refers to a disease, disorder, condition, or symptom of any of the foregoing.

### Engineering Tissue Constructs

It is well known that chronic diseases, among which cardiovascular diseases remain the principal cause of death in the Western civilization, often lead to organ failure. When this happens, the only solution is tissue replacement. Unfortunately, there is a near constant shortage of organs for transplantation and many subjects in need of an organ transplant never receive one. Tissue engineering could, in principle, provide unlimited material for surgery and ultimately an unlimited supply of custom-made organs suitable for transplantation. Notable proofs of concept of functional constructs obtained by different methods have been demonstrated. The limitation of this approach remains scalability: large-volume tissues need rapid, preferably automatic, and reproducible cellular assembly. A bottleneck faced by tissue engineering is the difficulty to provide engineered tissue with vascularization and innervation.

Biofabrication has both the precision and the speed to create large bio-similar structures with clinical applicability, potentially from patient-specific cells. One of the most advanced biofabrication methods is bioprinting. Traditionally, bioprinters require a cell-embedding scaffold biomaterial, typically called “bioink.” Bioprinting depends on bioinks or scaffolds to provide mechanical support to the cells during their deployment in a predefined, anatomically meaningful 3D structure. It can be difficult to find scaffolds capable of serving both as a good printing material and as a suitable environment for the contained cells. Moreover, the interposition of the bioink between cells during and after bioprinting limits the cells' ability to communicate and to optimally perform their biological functions.

In bioprinting, the biomaterial's suitability for dispersion by the bioprinter and its role as biological support must be taken into consideration. Depending on the type of bioprinting desired (ink-jet, extrusion, laser-assisted, etc.), there are different reasons for which the efficiency of scaffold-dependent bioprinting remains low, and its anticipated translation to clinic remains uncertain. For at least these reasons, scaffold-dependent bioprinting methods have clear disadvantages.

Scaffold-free biofabrication (i.e., without the use of embedded biomaterials) uses only cells and their own extracellular matrix for creating bio-similar constructs. Because individual cells are very small, it is useful to prepare them prior to biofabrication applications in pre-assembled cell clusters called “spheroids.” This pre-assembly also gives the cells the conditions to engage in phenotype-appropriate interactions and to secrete their own matrix.

One such scaffold-free bioprinting method is the ‘Kenzan’ method which laces and temporarily keeps clusters of cells/spheroids onto microneedle arrays. These are thus used as temporary mechanical supports, until the cell clusters/spheroids adhere to each other or are incorporated into a larger structure (e.g., the 3D bio-assembling robot Regenova™, manufactured by Cyfuse Biomedical, Tokyo, Japan). The use of spheroids with this method can therefore generate larger engineered tissue structures, such as cardiac patches, vascular tubes, trachea, urethra, etc. However, finding the cells capable of producing appropriate cell clusters/spheroids with suitable properties for scaffold-free biofabrication, and for use in the Kenzan method, remains a largely empirical undertaking that requires significant human intervention. The Kenzan method and a device useful in Kenzan bioprinting are described in Moldovan, N. I., Hibino, N., and Nakayama, K., Principles of the Kenzan Method of Robotic Cell Spheroid-Based Three Dimensional Bioprinting, Tissue Eng Part B Rev., 23(3):237-244 (2017) and U.S. Pat. No. 9,441,194.

### Pluripotent Stem Cells

Pluripotent stem cells have the ability to give rise to progeny that can undergo differentiation, under the appropriate conditions, into cell types from all three embryonic germ layers (endoderm, mesoderm, and ectoderm) from which all tissues and organs derive. The endoderm is the source of, for example, pharynx, esophagus, stomach, intestine and associated glands (e.g., salivary glands), liver, epithelial linings of respiratory passages and gastrointestinal tract, pancreas and lungs. The mesoderm is the source of, for example, smooth and striated muscle, connective tissue, blood vessels, the cardiovascular system, blood cells, endothelial cells, bone marrow, skeleton, reproductive organs and excretory organs. Ectoderm is the source of, for example, epidermis (epidermal layer of the skin), sensory organs, the entire nervous system, including brain, spinal cord, and all the outlying components of the nervous system.

Thus, pluripotent stem cells can contribute to many or if not all tissues of a prenatal, postnatal or adult animal. A standard art-accepted test, such as the ability to form a teratoma in 8-12-week-old SCID mice, can be used to establish the pluripotency of a cell population, however identification of various pluripotent stem cell characteristics can also be used to distinguish pluripotent cells from other cells. For example, the ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics.

For example, human pluripotent stem cells express at least some, and optionally all, of the markers from the following non-limiting list: SSEA-3, SSEA-4, TRA-1-60, TRA-1-81, TRA-2-49/6E, ALP, SOX2, E-CADHERIN, UTF-1, OCT4, REX1, AND NANOG.

Pluripotent stem cells suitable for use in the methods of the present disclosure are thus cells with unlimited self-renewal potential in culture including, for example, embryonic stem (ES) cells, primordial germ cells or induced pluripotent stem cells. In a preferred embodiment, pluripotent stem cells are cells that express at least one functional stem cell transcription factor, e.g., Oct-4A, SOX-2 or NANOG. A “functional” stem cell transcription factor does not include pseudogenes of OCT-4, SOX-2 or NANOG.

Examples of pluripotent cells include, but are not limited to, the human embryonic stem cell (hESC) line H9, fibroblast-derived human iPS cell line DF19-9-11T, hiPS cell line FCB-iPS-1; or hiPS cell line FCB-iPS-2, as described, for example, in the PCT publication WO 2015/138634.

In certain embodiments, the pluripotent stem cells can be induced pluripotent stem cells (iPSCs) that are generated by introducing a specific combination of stem cell transcription factors into a non-pluripotent cell (e.g. Oct-3/4, Sox2, KLF4 and c-Myc; see, Takahashi, K. & Yamanaka, S. Cell 126, 663-676 (2006); Okita, K. et al. Nature 448, 313-317 (2007); Wernig, M. et al. Nature 448, 318-324 (2007); Maherali, N. et al. Cell Stem Cell 1, 55-70 (2007); Meissner et al. Nature Biotechnol. 25, 1177-1181 (2007); Yu, J. et al. Science 318, 1917-1920 (2007); Nakagawa, M. et al. Nature Biotechnol. 26, 101-106 (2007); Wernig et al. Cell Stem Cell 2, 10-12 (2008)). iPSCs resemble thus pluripotent embryonic stem cells in their potential to differentiate into a full spectrum of adult somatic cell types and in their morphology, proliferation, gene expression, surface antigen expression, epigenetic status of pluripotent cell-specific genes, and telomerase activity.

In other embodiments, iPSCs can also be chemically induced from adult somatic cells (see, e.g. U.S. Pat. No. 9,394,524).

In still other embodiments, primary human skin fibroblasts can be reprogrammed into induced pluripotent stem cells (iPSCs) by gene editing of the endogenous OCT4, SOX2, KLF4, MYC, and LIN28A promoters and optionally a conserved Alu-motif enriched near genes involved in embryo genome activation (EEA-motif) (Weltner et al. (2018) Nature Communications volume 9, Article number: 2643).

Alternatively, induced pluripotent stem cell lines can be obtained from the ATCC, California Institute for Regenerative Medicine (CIRM) or European Bank for Induced Pluripotent Stem Cells as well as from commercial vendors.

Pluripotent cells are cultured under conditions suitable for maintaining pluripotent cells in an undifferentiated state. Methods for maintaining pluripotent cells in vitro, i.e., in an undifferentiated state, are well known in the art. For example, human ES and iPS cells may be maintained in mTeSR1 complete medium on Matrigel™ in 10 cm² tissue culture dishes at 37° C. and 5% CO2 for about two days. In certain embodiments, the culture medium contains leukemia inhibitory factor, or LIF, an interleukin 6 class cytokine that affects cell growth by inhibiting differentiation.

Additional and/or alternative methods for culturing and/or maintaining pluripotent cells may be used. For example, as the basal culture medium, any of TeSR, mTeSR1 αMEM, BME, BGJb, CMRL 1066, DMEM, Eagle MEM, Fischer's media, Glasgow MEM, Ham, IMDM, Improved MEM Zinc Option, Medium 199 and RPMI 1640, or combinations thereof, may be used for culturing and or maintaining pluripotent cells.

The pluripotent cell culture medium used may contain serum or it may be serum-free. Serum-free refers to a medium comprising no unprocessed or unpurified serum. Serum-free media can include purified blood-derived components or animal tissue-derived components, such as, for example, growth factors. The pluripotent cell medium used may contain one or more alternatives to serum, such as, for example, knockout Serum Replacement (KSR), chemically-defined lipid concentrated (Gibco) or Glutamax (Gibco).

Methods for passaging pluripotent cells are well known in the art. For example, after pluripotent cells are plated, the medium may be changed on days 2, 3, and 4 with cells being passaged on day 5. Generally, once a culture container is 70-100% confluent, the cell mass in the container is split into aggregated cells or single cells by any method suitable for dissociation and the aggregated or single cells are transferred into new culture containers. Cell “passaging” is a well-known technique for keeping cells alive and growing cells in vitro for extended periods of time.

### Endothelial Colony Forming Cells and Smooth Muscle Forming Cells

As used herein, the term “endothelial progenitor cell” refers to precursors of cells in the endothelial cell lineage. Examples of endothelial progenitor cells include, but are not limited to, colony-forming unit-Hill (CFU-Hill) cells, circulating angiogenic cells (CACs) and endothelial colony-forming cells (ECFCs). CFU-Hill cells and CACs are usually referred to as early outgrowth EPCs whereas ECFCs are termed as late outgrowth EPCs.

In certain embodiments, endothelial colony progenitor cells (EPCs) refer to endothelial colony forming cells (ECFCs) that are non-primary adult endothelial cells.

Properties of ECFCs include, but is not limited to, (A) characteristic ECFC molecular phenotype; (B) capacity to form capillary-like networks in vitro on Matrigel™; (C) high proliferation potential; (D) self-replenishing potential; (E) capacity for blood vessel formation in vivo without co-culture with any other cells; (F) increased cell viability and/or decreased senescence and (G) cobblestone morphology. Furthermore, as with ECFCs, the methods of generating ECFC cells described herein do not require co-culture with supportive cells, such as, for example, OP9 bone marrow stromal cells, embryoid body (EB) formation or exogenous TGF-β inhibition.

ECFCs are capable of differentiating to regenerate endothelial cell populations. They are customarily residents of adult vasculature and they can migrate to areas of injury as a circulating endothelial cell precursor. They play a major role in vascular healing after injury as well as in developmental angiogenesis. ECFCs of both cord and adult blood, or of pluripotent stem cell origin, are a very promising cell population for cardiovascular medicine. Because their in vivo applications for cell therapy face the same biological constraints as other single-cell suspensions, as demonstrated herein, it is advantageous to arrange them into cell clusters or spheroids. Experimentally ECFC progenitor cells can be defined as CD31⁺ and NRP1⁻ cells.

In certain embodiments, ECFCs can be derived from pluripotent stem cells such as iPSCs. Examples of methods for deriving ECFCs from iPSCs are described herein, as well as in Prasain, N. et al., Differentiation of Human Pluripotent Stem Cells Similar to Cord-Blood Endothelial Colony-Forming Cells, Nat Biotechnol, 32(11):1151-1157 (2014).

In some embodiments, a population of ECFCs is derived from peripheral blood. Examples of methods for deriving ECFCs from peripheral blood or cord blood are described, for example, in Melero-Martin, J. M., et al., In vivo Vasulogenic Potential of Human Blood-Derived Endothelial Progenitor Cells, Blood, 109:4761-4768 (2007).

Smooth-muscle forming cells initially express CD31 on their cell surface, but not NRP1. Upon further differentiation, SMFCs express the alpha isoform of smooth-muscle-type actin. These biomarkers suggest that CD31⁺NRP1⁺ cells are progenitors of other cell types such as vascular smooth muscle cells and/or myo-fibroblasts. Examples of methods for deriving SMFCs from iPSCs are described herein. Experimentally, SMFC progenitor cells can be defined as CD31⁺NRP1⁻ cells.

Given the roles in vascular biology and tissue repair of ECFC and SMFC iPSC-derived cell populations, their use in engineered tissue constructs, particularly as cell-clusters, has great therapeutic potential. As further described herein, the present disclosure details a method capable of the production of large quantities of ECFCs and SMFCs from iPSCs. The present disclosure also provides methods for the large-scale amplification (more than five orders of magnitude) of iPSC-derived ECFCs and SMFCs, production of spheroids generated from iPSC-derived ECFCs and SMFCs, and engineered tissue constructs comprising these specialized spheroids.

In some embodiments, iPSC-derived ECFCs and SMFCs can be selected and isolated by fluorescence-activated cell sorting (FACS) of double-positive cells, based on the expression of CD31 (PECAM-1), a cell surface molecule involved in homotypic interaction between the vascular endothelium and the circulating leukocytes, and Neuropilin-1 (NRP1), a coreceptor with VEGF receptor type 2 for the endothelial vascular growth factor (VEGF), involved in endothelial signaling. During this selection process, CD31⁺NRP1⁺ cells (i.e. ECFC progenitor cells) were identified as well as a population of CD31 positive iPSC-derived cells that were NRP1 negative. Unlike the double positive CD31⁺NRP1⁺ cells, CD31⁺NRP1⁻ cells further differentiate to express the alpha isoform of smooth-muscle-type actin, indicating that these cells are likely progenitors of vascular smooth muscle cells and/or myofibroblasts. Methods for differentiating iPSCs and selecting ECFC progenitor cells are described in Prasain, N. et al., Differentiation of Human Pluripotent Stem Cells Similar to Cord-Blood Endothelial Colony-Forming Cells, Nat Biotechnol, 32(11):1151-1157 (2014).

By a “CD31⁺ cell” is meant a cell that expresses a detectable level of CD31 polypeptide, polynucleotide, or fragment thereof. CD31 is a cell adhesion molecule which is required for leukocyte transendothelial migration (TEM) under most inflammatory conditions. Tyr-690 plays a major role in TEM and is required for efficient trafficking of PECAM1 to and from the lateral border recycling compartment (LBRC) and is also essential for the LBRC membrane to be targeted around migrating leukocytes. Trans-homophilic interaction may play a role in endothelial cell-cell adhesion via cell junctions. PECAM also modulates bradykinin receptor BDKRB2 activation as well as bradykinin- and hyperosmotic shock-induced ERK1/2 activation in endothelial cells.

“CD31” (also called platelet/endothelial cell adhesion molecule or PECAM, EndoCAM, GPIIA, PECA) refers to a polypeptide, protein or fragment thereof having at least 85% identity to the amino acid sequence provided at NP_000433.

In certain embodiments, human CD31 comprises the cDNA sequence (NM 000442) having the DNA sequence of SEQ ID NO: 2 and amino acid sequence of SEQ ID NO: 1 (NP_000433) as shown in TABLE I below:

TABLE I

| SEQ ID | | | |
|---|---|---|---|
| NO | | CD31 cDNA SEQUENCE (coding region) | |
| 1 | 1 | M Q P R W A Q G A T M W L G V L L T L L | 20 |
| 2 | 1 | atgcagccgaggtgggcccaaggggccacgatgtggcttggagtcctgctgacccttctg | 60 |
| | 21 | L C S S L E G Q E N S F T I N S V D M K | 40 |
| | 61 | ctctgttcaagccttgagggtcaagaaaactctttcacaatcaacagtgttgacatgaag | 120 |
| | 41 | S L P D W T V Q N G E N L T L Q C F A D | 60 |
| | 121 | agcctgccggactggacggtgcaaaatgggaagaacctgaccctgcagtgcttcgcggat | 180 |
| | 61 | V S T T S H V K P Q H Q M L F Y K D D V | 80 |
| | 181 | gtcagcaccacctctcacgtcaagcctcagcaccagatgctgttctataaggatgacgtg | 240 |
| | 81 | L F Y N I S S M K S T E S Y F I P E V R | 100 |
| | 241 | ctgttttacaacatctcctccatgaagagcacagagagttattttattcctgaagtccgg | 300 |
| | 101 | I Y D S G T Y K C T V I V N N K E K T T | 120 |
| | 301 | atctatgactcagggacatataaatgtactgtgattgtgaacaacaaagagaaaaccact | 360 |
| | 121 | A E Y Q V L V E G V P S P R V T L D K K | 140 |
| | 361 | gcagagtaccaggtgttggtggaaggagtgcccagtcccagggtgacactggacaagaaa | 420 |
| | 141 | E A I Q G G I V R V N C S V P E E K A P | 160 |
| | 421 | gaggccatccaaggtgggatcgtgagggtcaactgttctgtcccagaggaaaaggcccca | 480 |
| | 161 | I H F T I E K L E L N E K M V K L E R E | 180 |
| | 481 | atacacttcacaattgaaaaacttgaactaaatgaaaaaatggtcaagctgaaaagagag | 540 |
| | 181 | K N S R D Q N F V I L E F P V E E Q D R | 200 |
| | 541 | aagaattctcgagaccagaattttgtgatactggaattccccgttgaggaacaggaccgc | 600 |
| | 201 | V L S F R C Q A R I I S G I H M Q T S E | 220 |
| | 601 | gttttatccttccgatgtcaagctaggatcatttctgggatccatatgcagacctcagaa | 660 |
| | 221 | S T E S E L V T V T E S F S T P K F H I | 240 |
| | 661 | tctaccaagagtgaactggtcaccgtgacggaatccttctctacacccaagttccacatc | 720 |
| | 241 | S P T G M I M E G A Q L H I E C T I Q V | 260 |
| | 721 | agccccaccggaatgatcatggaaggagctcagctccacattaagtgcaccattcaagtg | 780 |
| | 462 | T H L A Q E F P E I I I Q K D K A I V A | 280 |
| | 781 | actcacctggcccaggagtttccagaaatcataattcagaaggacaaggcgattgtggcc | 840 |
| | 281 | H N R H G N K A V Y S V M A M V E H S G | 300 |
| | 841 | cacaacagacatggcaacaaggctgtgtactcagtcatggccatggtggagcacagtggc | 900 |
| | 301 | N Y T C K V E S S R I S K V S S I V V N | 320 |
| | 901 | aactacacgtgcaaagtggagtccagccgcatatccaaggtcagcagcatcgtggtcaac | 960 |
| | 321 | I T E L F S K P E L E S S F T H L D Q G | 340 |
| | 961 | ataacagaactattttccaagcccgaactggaatcttccttcacacatctggaccaaggt | 1020 |
| | 341 | E R L N L S C S I P G A P P A N F T I Q | 360 |
| | 1021 | gaaagactgaacctgtcctgctccatcccaggagcacctccagccaacttcaccatccag | 1080 |
| | 361 | K E D T I V S Q T Q D F T K I A S K S D | 380 |
| | 1081 | aaggaagatacgattgtgtcacagactcaagatttcaccaagatagcctcaaagtcggac | 1140 |
| | 381 | S G T Y I C T A G I D E V V E K S N T V | 400 |
| | 1141 | agtgggacgtatatctgcactgcaggtattgacaaagtggtcaagaaaagcaacacagtc | 1200 |
| | 401 | Q I V V C E M L S Q P R I S Y D A Q F E | 420 |
| | 1201 | cagatagtcgtatgtgaaatgctctcccagcccaggatttcttatgatgcccagtttgag | 1260 |
| | 421 | V I K G Q T I E V R C E S I S G T L P I | 440 |
| | 1261 | gtcataaaaggacagaccatcgaagtccgttgcgaatcgatcagtggaactttgcctatt | 1320 |
| | 441 | S Y Q L L E T S K V L E N S T E N S N D | 460 |
| | 1321 | tcttaccaacttttaaaaacaagtaaagttttggagaatagtaccaagaactcaaatgat | 1380 |
| | 461 | P A V F E D N P T E D V E Y Q C V A D N | 480 |
| | 1381 | cctgcggtattcaaagacaaccccactgaagacgtcgaataccagtgtgttgcagataat | 1440 |
| | 481 | C H S H A K M L S E V L R V K V I A P V | 500 |
| | 1441 | tgccattcccacgccaaaatgttaagtgaggttctgagggtgaaggtgatagccccggtg | 1500 |
| | 501 | D E V Q I S I L S S K V V E S G E D I V | 520 |
| | 1501 | gatgaggtccagatttctatcctgtcaagtaaggtggtggagtctggagaggacattgtg | 1560 |
| | 521 | L Q C A V N E G S G P I T Y K F Y R E K | 540 |
| | 1561 | ctgcaatgtgctgtgaatgaaggatctggtcccatcacctataagttttacagagaaaaa | 1620 |
| | 541 | E G K P F Y Q M T S N A T Q A F W T K Q | 560 |
| | 1621 | gagggcaaacccttctatcaaatgacctcaaatgccacccaggcattttggaccaagcag | 1680 |
| | 561 | K A N K E Q E G E Y Y C T A F N R A N H | 580 |
| | 1681 | aaggctaacaaggaacaggagggagagtattactgcacagccttcaacagagccaaccac | 1740 |
| | 581 | A S S V P R S K I L T V R V I L A P W K | 600 |
| | 1741 | gcctccagtgtccccagaagcaaaatactgacagtcagagtcattcttgccccatggaag | 1800 |
| | 601 | K G L I A V V I I G V I I A L L I I A A | 620 |
| | 1801 | aaaggacttattgcagtggttatcatcggagtgatcattgctctcttgatcattgcggcc | 1860 |
| | 621 | K C Y F L R K A K A K Q M P V E M S R P | 640 |
| | 1861 | aaatgttattttctgaggaaagccaaggccaagcagatgccagtggaaatgtccaggcca | 1920 |
| | 641 | A V P L L N S N N E K M S D P N M E A N | 660 |
| | 1921 | gcagtaccacttctgaactccaacaacgagaaaatgtcagatcccaatatggaagctaac | 1980 |
| | 661 | S H Y G H N D D V G N H A M K P I N D N | 680 |
| | 1981 | agtcattacggtcacaatgacgatgtcggaaaccatgcaatgaaaccaataaatgataat | 2040 |
| | 681 | K E P L N S D V Q Y T E V Q V S S A E S | 700 |
| | 2041 | aaagagcctctgaactcagacgtgcagtacacggaagttcaagtgtcctcagctgagtct | 2100 |
| | 701 | H K D L G K K D T E T V Y S E V R K A V | 721 |
| | 2101 | cacaaagatctaggaaagaaggacacagagacagtgtacagtgaagtccggaaagctgtc | 2160 |
| | 721 | P D A V E S R Y S R T E G S L D G T * | 740 |
| | 2161 | cctgatgccgtggaaagcagatactctagaacggaaggctcccttgatggaacttag | 2220 |

“Neuropilin-1” (also referred to as Neuropilin, Vascular Endothelial Cell Growth Factor 165 Receptor, VEGF165R, NRP, Transmembrane Receptor 3, CD304 Antigen, BDCA4, CD304 or NP1) refers to a polypeptide, protein or fragment thereof having at least 85% identity to the amino acid sequence provided at NP_000433.

Neuropilin-1 contains specific protein domains which allow it to participate in several different types of signaling pathways that control cell migration. Neuropilins contain a large N-terminal extracellular domain, made up of complement-binding, coagulation factor V/VIII, and meprin domains. These proteins also contain a short membrane-spanning domain and a small cytoplasmic domain. Neuropilins bind many ligands and various types of co-receptors; they affect cell survival, migration, and attraction. Some of the ligands and co-receptors bound by neuropilins are vascular endothelial growth factor (VEGF) and semaphorin family members. Several alternatively spliced transcript variants that encode different protein isoforms have been reported.

In certain embodiments, human neuropilin-1 (NRP-1) comprises the cDNA sequence (NM_003873) having the DNA sequence of SEQ ID NO: 3 and amino acid sequence (NP_003864) of SEQ ID NO: 4 as shown in TABLE II below:

TABLE II

| SEQ ID | | | |
|---|---|---|---|
| NO | | NEUROPILIN-1 cDNA SEQUENCE (coding region) | |
| 3 | 1 | M E R G L P L L C A V L A L V L A P A G | 20 |
| 4 | 1 | atggagagggggctgccgctcctctgcgccgtgctcgccctcgtcctcgccccggccggc | 60 |
| | 21 | A F R N D K C G D T I K I E S P G Y L T | 40 |
| | 61 | gcttttcgcaacgataaatgtggcgatactataaaaattgaaagccccgggtaccttaca | 120 |
| | 41 | S P G Y P H S Y H P S E K C E W L I Q A | 60 |
| | 121 | tctcctggttatcctcattcttatcacccaagtgaaaaatgcgaatggctgattcaggct | 180 |
| | 61 | P D P Y Q R I M I N F N P H F D L E D R | 80 |
| | 181 | ccggacccataccagagaattatgatcaacttcaaccctcacttcgatttggaggacaga | 240 |
| | 81 | D C K Y D Y V E V F D G E N E N G H F R | 100 |
| | 241 | gactgcaagtatgactacgtggaagtcttcgatggagaaaatgaaaatggacattttagg | 300 |
| | 101 | G K F C G K I A P P P V V S S G P F L F | 120 |
| | 301 | ggaaagttctgtggaaagatagcccctcctcctgttgtgtcttcagggccatttcttttt | 360 |
| | 121 | I K F V S D Y E T H G A G F S I R Y E I | 140 |
| | 361 | atcaaatttgtctctgactacgaaacacatggtgcaggattttccatacgttatgaaatt | 420 |
| | 141 | F K R G [ E C S Q M Y T T P S G V I K S | 160 |
| | 421 | ttcaagagaggtcctgaatgttcccagaactacacaacacctagtggagtgataaagtcc | 480 |
| | 161 | P G F P E K Y P N S L E C T Y I V F A P | 180 |
| | 481 | cccggattccctgaaaaatatcccaacagccttgaatgcacttatattgtctttgcgcca | 540 |
| | 181 | K M S E I I L E F E S F D L E P D S N P | 200 |
| | 541 | aagatgtcagagattatcctggaatttgaaagctttgacctggagcctgactcaaatcct | 600 |
| | 201 | P G G M F C R Y D R L E I W D G F P D V | 220 |
| | 601 | ccaggggggatgttctgtcgctacgaccggctagaaatctgggatggattccctgatgtt | 660 |
| | 221 | G P H I G R Y C G Q K T P G R I R S S S | 240 |
| | 661 | ggccctcacattgggcgttactgtggacagaaaacaccaggtcgaatccgatcctcatcg | 720 |
| | 241 | G I L S M V F Y T D S A I A K E G F S A | 260 |
| | 721 | ggcattctctccatggttttttacaccgacagcgcgatagcaaaagaaggtttctcagca | 780 |
| | 261 | N Y S V L Q S S V S E D F K C M E A L G | 280 |
| | 781 | aactacagtgtcttgcagagcagtgtctcagaagatttcaaatgtatggaagctctgggc | 840 |
| | 281 | M E S G E I H S D Q I T A K K Q Y S T N | 300 |
| | 841 | atggaatcaggagaaattcattctgaccagatcacagcttcttcccagtatagcaccaac | 900 |
| | 301 | W S A E R S R L N Y P E N G W T P G E D | 320 |
| | 901 | tggtctgcagagcgctcccgcctgaactaccctgagaatgggtggactcccggagaggat | 960 |
| | 321 | S Y R E W I Q V D L G L L R F V T A V G | 340 |
| | 961 | tcctaccgagagtggatacaggtagacttgggccttctgcgctttgtcacggctgtcggg | 1020 |
| | 341 | T Q G A I S K E T K K K Y Y V K T Y K I | 360 |
| | 1021 | acacagggcgccatttcaaaagaaaccaagaagaaatattatgtcaagacttacaagatc | 1080 |
| | 361 | D V S S N G E D W I T I K E G N K P V L | 380 |
| | 1081 | gacgttagctccaacggggaagactggatcaccataaaagaaggaaacaaacctgttctc | 1140 |
| | 381 | F Q G N T N P T D V V V A V F P K P L I | 400 |
| | 1141 | tttcagggaaacaccaaccccacagatgttgtggttgcagtattccccaaaccactgata | 120 |
| | 401 | T R F V R I K P A T W E T G I A M R F E | 420 |
| | 1201 | actcgatttgtccgaatcaagcctgcaacttgggaaactggcatatctatgagatttgaa | 1260 |
| | 421 | V Y G C K I T D Y P C S G M L G M V S G | 440 |
| | 1261 | gtatacggttgcaagataacagattatccttgctctggaatgttgggtatggtgtctgga | 1320 |
| | 441 | L I S D S Q I T S S N Q G D R N W M P E | 460 |
| | 1321 | cttatttctgactcccagatcacatcatccaaccaaggggacagaaactggatgcctgaa | 1380 |
| | 461 | N I R L V T S R S G W A L P P A P H S Y | 480 |
| | 1381 | aacatccgcctggtaaccagtcgctctggctgggcacttccacccgcacctcattcctac | 1440 |
| | 481 | I N E W L Q I D L G E E K I V R G I I I | 500 |
| | 1441 | atcaatgagtggctccaaatagacctgggggaggagaagatcgtgaggggcatcatcatt | 1500 |
| | 501 | Q G G K H R E N K V F M R K F K I G Y S | 520 |
| | 1501 | cagggtgggaagcaccgagagaacaaggtgttcatgaggaagttcaagatcgggtacagc | 1560 |
| | 521 | N N G S D W K M I M D D S K R K A K S F | 540 |
| | 1561 | aacaacggctcggactggaagatgatcatggatgacagcaaacgcaaggcgaagtctttt | 1620 |
| | 541 | E G N N N Y D T P E L R T F P A L S T R | 561 |
| | 1621 | gagggcaacaacaactatgatacacctgagctgcggacttttccagctctctccacgcga | 1680 |
| | 561 | F I R I Y P E R A T H G G L G L R M E L | 580 |
| | 1681 | ttcatcaggatctaccccgagagagccactcatggcggactggggctcagaatggagctg | 1740 |
| | 581 | L G C E V E A P T A G P T T P N G N L V | 600 |
| | 1741 | ctgggctgtgaagtggaagcccctacagctggaccgaccactcccaacgggaacttggtg | 1800 |
| | 601 | D E C D D D Q A N C H S G T G D D F Q L | 620 |
| | 1801 | gatgaatgtgatgacgaccaggccaactgccacagtggaacaggtgatgacttccagctc | 1860 |
| | 621 | T G G T T V L A T E K P T V I D S T I Q | 641 |
| | 1861 | acaggtggcaccactgtgctggccacagaaaagcccacggtcatagacagcaccatacaa | 1920 |
| | 641 | S E F P T Y G F N C E F G W G S H K T F | 660 |
| | 1921 | tcagagtttccaacatatggttttaactgtgaatttggctggggctctcacaagaccttc | 1980 |
| | 661 | C H W E H D N H V Q L K W S V L T S K T | 680 |
| | 1981 | tgccactgggaacatgacaatcacgtgcagctcaagtggagtgtgttgaccagcaagacg | 2040 |
| | 681 | G P I Q D H T G D G N F I Y S Q A D E N | 700 |
| | 2041 | ggacccattcaggatcacacaggagatggcaacttcatctattcccaagctgacgaaaat | 2100 |
| | 701 | Q K G K V A R L V S P V V Y S Q N S A H | 720 |
| | 2101 | cagaagggcaaagtggctcgcctggtgagccctgtggtttattcccagaactctgcccac | 2160 |
| | 721 | C M T F W Y H M S G S H V G T L R V K L | 740 |
| | 2161 | tgcatgaccttctggtatcacatgtctgggtcccacgtcggcacactcagggtcaaactg | 2220 |
| | 741 | R Y Q K P E E Y D Q L V W M A I G H Q G | 760 |
| | 2221 | cgctaccagaagccagaggagtacgatcagctggtctggatggccattggacaccaaggt | 2280 |
| | 761 | D H W K E G R V L L H K S L K L Y Q V I | 780 |
| | 2281 | gaccactggaaggaagggcgtgtcttgctccacaagtctctgaaactttatcaggtgatt | 2340 |
| | 781 | F E G E I G K G N L G G I A V D D I S I | 800 |
| | 2341 | ttcgagggcgaaatcggaaaaggaaaccttggtgggattgctgtggatgacattagtatt | 2400 |
| | 801 | N N H I S Q E D C A K P A D L D K K N P | 820 |
| | 2401 | aataaccacatttcacaagaagattgtgcaaaaccagcagacctggataaaaagaaccca | 2460 |
| | 821 | E I K I D E T G S T P G Y E G E G E G D | 840 |
| | 2461 | gaaattaaaattgatgaaacagggagcacgccaggatacgaaggtgaaggagaaggtgac | 2520 |
| | 841 | K N I S R K P G N V L K T L D P I L I T | 860 |
| | 2521 | aagaacatctccaggaagccaggcaatgtgttgaagaccttagaccccatcctcatcacc | 2580 |
| | 861 | I I A M S A L G V L L G A V C G V V L Y | 880 |
| | 2581 | atcatagccatgagtgccctgggggtcctcctgggggctgtctgtggggtcgtgctgtac | 2640 |
| | 881 | C A C W H N G M S E R N L S A L E N Y N | 900 |
| | 2641 | tgtgcctgttggcataatgggatgtcagaaagaaacttgtctgccctggagaactataac | 2700 |
| | 901 | F E L V D G V K L K K D K L N T Q S T Y | 920 |
| | 2701 | tttgaacttgtggatggtgtgaagttgaaaaaagacaaactgaatacacagagtacttat | 2760 |
| | 921 | S E A * | 924 |
| | 2761 | tcggaggcatga | 2772 |

### Spheroids

Cell clusters are ubiquitous in both nature and biotechnology, as the cells perform their functions in direct or distant interaction with each other. An embryo—the starting unit of any organism—is essentially a cell cluster. Artificial “organoids” are also a form of cell spheroids that recapitulate tissue development, thus opening excellent opportunities for regenerative medicine.

Pre-assembled cell clusters, called “spheroids” due to their spherical shape, are increasingly used for a variety of applications, from 3D normal or pathological tissue models to biofabrication. In particular, injection of pre-formed cell spheroids into a subject has been shown to increase cell survival and efficiency of pro-angiogenic cell therapy with both primitive and adult cells, as compared to single-cell suspensions. This holds true for cord-blood mesenchymal cells, as well as for ECFCs, a sub-class of circulating endothelial progenitor cells that are capable of forming colonies in vitro.

Spheroid shape and size represent examples of defining properties for many tissue engineering applications. Diffusion of oxygen and nutrients depend on spheroid size, thus impacting on cell survival and differentiation. For example, in biofabrication methods such as Kenzan bioprinting, the spheroids should ideally be as close to spherical as possible in order to precisely fit the mouth of the nozzle which takes them from the formatting wells. Also, to come in contact with each other for subsequent fusion, these spheroids need to be uniform in size and with a diameter comparable with the distance between micro-needles.

Spheroids made from a single cell type, such as human umbilical endothelial cells-only spheroids, have been previously constructed. However, such spheroids are generally flawed for tissue engineering purposes because, while the cells on the surface survive, those at the spheroid's core eventually die by anoikis (i.e., apoptosis induced by the lack of attachment to a solid substrate). In that regard, the size and shape of spheroids can be a quality check on a bioprinter; those that do not conform to the size and shape standards required for the desired form of bioprinting can be set aside and not used, whereas those conforming to the size and shape parameters of the desired form of bioprinting can be used. As such, in some embodiments provided by the present disclosure, it is advantageous to determine the time it takes in culture for cell spheroids to attain a reasonably stable diameter prior to attempting biofabrication. If the spheroids are used too early, they might not be fully formed (in terms of intracellular interactions and extracellular matrix composition), and might not be robust enough to survive subsequent manipulation during tissue engineering. Alternatively, keeping them too long in culture could make them vulnerable to hypoxia and nutrient depletion at the core.

In various aspects, the present disclosure provides spheroids containing a combination of ECFC and SMFCs, as well as a specific maturation protocol (long-term incubation in endothelial cell-supporting medium) which leads to the formation of stable spheroids capable of use in a wide variety of tissue engineering applications. Additionally, in various aspects the disclosed spheroids are novel tissue cellular constructs for tissue engineering and/or cellular therapy in that they contain a pre-formed capillary-like structure at their core, surrounded by a protective cellular shell or coating. The disclosed spheroids therefore contain a microvasculature (MV) and can also be referred to as MV-containing spheroids.

In various aspects, the present disclosure provides robust, pre-formed, MV-containing spheroids formed from SMFCs which maintain their structural durability even when up to 20% of each individual spheroid is replaced by ECFCs. Further, the inventors have discovered that SMFCs appear to provide protection to the ECFCs in the spheroids. Additionally, the SMFCs relocate to the core of the disclosed spheroids, where they differentiate into vascular cords/microvasculature. The present disclosure also provides engineered tissue constructs comprising pre-formed MV-containing spheroids, wherein the pre-formed MV-containing spheroids survived and reorganized after an extended in vitro post-printing maturation, and after in vivo implantation.

In certain embodiments, the present disclosure provides a method for producing pre-formed MV-containing spheroids. In certain embodiments, the method comprises seeding (i.e., culturing) ECFCs and SMFCs together, and culturing the mixture of cells together, allowing the different types of cells to contact each other. In some embodiments, both the ECFCs and SMFCs are derived from pluripotent stem cell, e.g. induced pluripotent stem cells (iPSCs). In certain embodiments, the SMFCs are iPSC-derived. In certain embodiments, the ECFCs are derived from peripheral blood, cord blood, iPSCs, or a combination of these sources.

In certain embodiments, the method comprises seeding ECFCs and SMFCs in an appropriate culture medium for an appropriate period of time. During the appropriate period of time, the cells self-aggregate into the disclosed spheroids. Additionally, the cells may self-sort, such that one type of cell becomes more concentrated in a particular region of the spheroid. Such self-sorting may occur during spheroid formation or after spheroid formation.

In certain embodiments, ECFCs and SMFCs are seeded as a mixture comprising about 0.5% to about 20% ECFCs and about 80% to about 99.5% SMFCs. In certain embodiments, ECFCs and SMFCs are seeded as a mixture comprising about 20% ECFCs and about 80% SMFCs.

Also provided is a method for producing ECFC spheroids. In some embodiments the method comprises seeding ECFC in an appropriate culture medium for an appropriate period of time. During the appropriate period of time, the ECFC self-aggregate into spheroids. In certain embodiments, the ECFC are derived from peripheral blood, cord blood, pluripotent stem cells, e.g., iPSCs, or a combination of these sources.

Also provided is a method for producing SMFC spheroids. In some embodiments the method comprises seeding SMFC in an appropriate culture medium for an appropriate period of time. During the appropriate period of time, the SMFC self-aggregate into spheroids. In certain embodiments, the SMFC are derived from pluripotent stem cells, e.g. iPSCs.

In certain embodiments, cells are seeded at a concentration of about 1.0×10⁴ cells/well to about 1.0×10⁶ cells/well. In certain embodiments, cells are seeded at 2.5×10⁴ cells/well.

In certain embodiments, the cells are cultured for between 12 and 72 hours prior to spheroid collection, with spheroid formation occurring in 18-24 hours of this culture time. In certain embodiments, the cells are cultured for 4 hours prior to spheroid collection. In certain embodiments, the cells are cultured for 8 hours prior to spheroid collection. In certain embodiments, in certain embodiments, the cells are cultured for 12 hours prior to spheroid collection. In certain embodiments, in certain embodiments, the cells are cultured for 16 hours prior to spheroid collection. In certain embodiments, the cells are cultured for 20 hours prior to spheroid collection. In certain embodiments, the cells are cultured for 24 hours or more prior to spheroid collection.

In certain embodiments, the culturing is performed in ultra-low adhesive U-bottomed well plates. In some embodiments, the ultra-low adhesive U-bottomed plates are 96 well plates.

In some embodiments, the appropriate culture medium is an endothelial cell-supporting medium. In some embodiments, the medium is EGM-2 medium.

In certain embodiments, spheroids can be formed using the hanging drop culture method. (see Kelm and Fussenegger, 2004, Trends in Biotechnology Vol. 22, No. 4: 195-202). Drops of cell culture medium with suspended cells are placed onto a cell culture surface and the plate is inverted. As there is no substrate available on which the cells can adhere, they accumulate at the bottom of the drop and form a spheroid. The technique can be scaled up by using, for example, IMAPlateTM5RC96 which is marketed by NCL New Concept Lab Gmb, Moehlin, Switzerland. An advantage of the hanging drop method is that it produces spheroids of uniform size and having a precise cellular composition. The procedure also avoids spheroids from adhering to cell culture dishes or coalescing together. Hanging drop cultures methods including methods of making spheroid tissue microarrays are described, for example, in U.S. Pat. Nos. 9,126,199; 8,906,685; 8,980,631 and International Patent Application Nos. WO2015069742 and WO2017174955.

In certain embodiments, spheroids can be formed by culturing the cells in media comprising polymers such as methylcellulose, carboxymethylcellulose, hydroxymethylcellulose, hydroxyethylcellulose, hydroxypropylcellulose or hydroxypropylmethylcellulose as described, for example, in International Patent Application Nos. WO2009111022 and WO2016090361.

The method provided by the present disclosure can be used to produce three types of cell spheroids: ECFC only spheroids; SMFC only spheroids; and ECFC+SFMC spheroids. As disclosed herein, all three spheroids types are capable of attaining a stable size in about 18-24 hours, with much of their aggregation occurring as early as 3 hours. This is substantially faster than the same time required for certain other cell types, which for the same purpose can take several days.

During the culturing step, even in a short interval, the cell ratio within some of the disclosed spheroids significantly changes during spheroid formation. Surprisingly, cell loss occurs in spheroids made of only ECFCs. Without wishing to be bound by any particular theory, it is presently anticipated that the growth medium cannot account for this effect, given that it was optimized for ECFC cultivation, and that SMFC only spheroids are viable in this medium. Without wishing to be bound by any particular theory, a possible explanation for this observed effect is that 3D aggregation and compaction is an non-physiological situation for the endothelial phenotype; combining poor oxygenation and nutrient depletion, the compaction collectively results in an ischemic condition, routinely found at the core of spheroids larger than 400 microns (that 200 microns is the limit of oxygenation diffusion in vivo).

Further, in the context of the disclosed method, SMFCs protect the ECFCs against decay in the spheroids. These SMFCs may either: i) provide an environment rich in supporting paracrine growth factors; or ii) secrete an extracellular matrix that permits the ECFCs to penetrate and/or organize in pre-vascular cords, thus promoting their maturation. At the same time, adding ECFCs in an amount up to 20% of the total amount of SMFCs in a given spheroid did not change the dynamics of the SMFCs cells in these mixed spheroids, confirming the structural and biological robustness of SMFCs.

In mixed spheroids (containing more than one type of cell), the final cell distribution resulting from cell sorting depends on biophysical parameters such as the strength of inter-cellular interactions. However, cell migration following chemotactic gradients is also likely to occur. This may explain the observation that, within a single spheroid produced by the disclosed method, ECFCs surprisingly accumulate at the spheroid's core. ECFCs in culture form disperse, unstructured colonies that give rise to loosely aggregated spheroids. Thus, ECFCs were expected to have a reduced intercellular attachment, primarily because of their localization at the periphery of the spheroids. The observed result is thus the opposite of what was expected. Without wishing to be bound by any particular theory, it is presently believed that this polarization of migration could be the result of ECFCs chemo-tropism towards the hypoxic core, likely mediated by a VEGF concentration gradient. Once located at the core, ECFCs may undergo further maturation under the influence of VEGF. Such a gradient would also explain the apparent sprouting from the ECFCs cords, a characteristic of an actively proliferating endothelium. This scenario is also consistent with the observed caspase 3 activity at the core where it could function both in apoptosis and in non-apoptotic differentiation.

Biofabrication of Spheroids Using iPSC-Derived Vascular Progenitor Cells

The present disclosure provides pre-formed MV-containing spheroids having properties suitable for use in scaffold-free biofabrication. Surprisingly, the mixed spheroids provide improved properties and viability at their core, encapsulating microvascular fragments which are ready for large scale assembly.

In certain embodiments, the pre-formed MV-containing spheroids can be used to assemble an engineered tissue using 3D bioprinting. An advantage of using a 3D bioprinter is the uniform and reproducible distribution of pre-formed MV-containing spheroids at defined locations within the tissue construct. In certain embodiments, other traditional bio-assembly or bioprinting methods can also be utilized.

In certain embodiments, in order to utilize biofabrication (for example, by the Kenzan method), in addition to selecting spheroids of a desired size and shape suitable for the biofabrication process, pre-formed MV-containing spheroids need to be sturdy enough for handling, both during uptake and piercing on the micro-needles. Spheroids containing ECFCs alone were observed to lack the requisite sturdiness. By contrast, spheroids comprising SMFCs either alone or in combination with ECFCs were sufficiently sturdy to withstand most forms of biofabrication. As noted above, these mixed cell type spheroids self-sort in a way that allows for the formation of microvasculature within their core. The observed sturdiness of the mixed cell type spheroids indicates that these spheroids can used as building blocks for scaffold-free biofabrication.

In certain embodiments, biofabricated engineered tissues (e.g., tubular constructs) using pre-formed MV-containing spheroids express alpha smooth muscle actin and collagen IV (a major component of the vascular basement membrane), thus corroborating their use as vascular grafts. In this regard, an unexpected finding was the layered distribution of smooth muscle actin towards both inner and outer surfaces of the tubular constructs. This could be the result of the construct's exposure to fluid shear stress in the bioreactor because biomechanical stress on vascular cells is known to induce the expression of this marker in vivo.

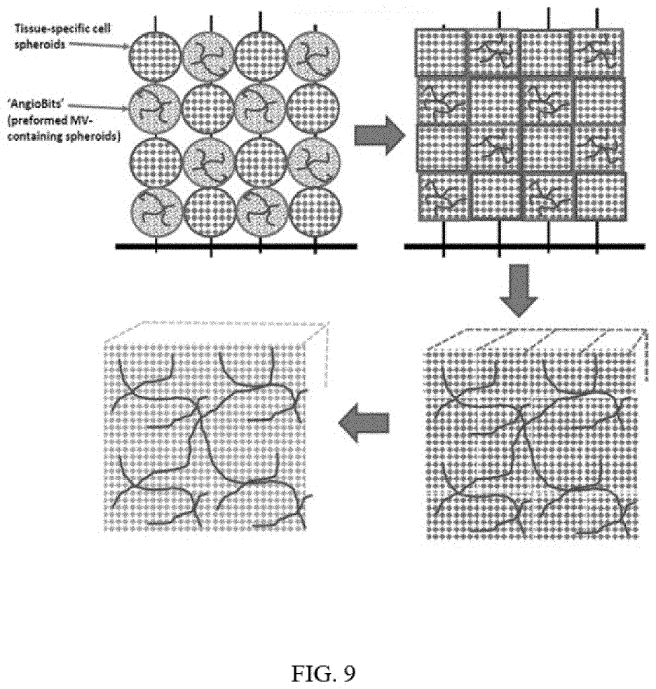

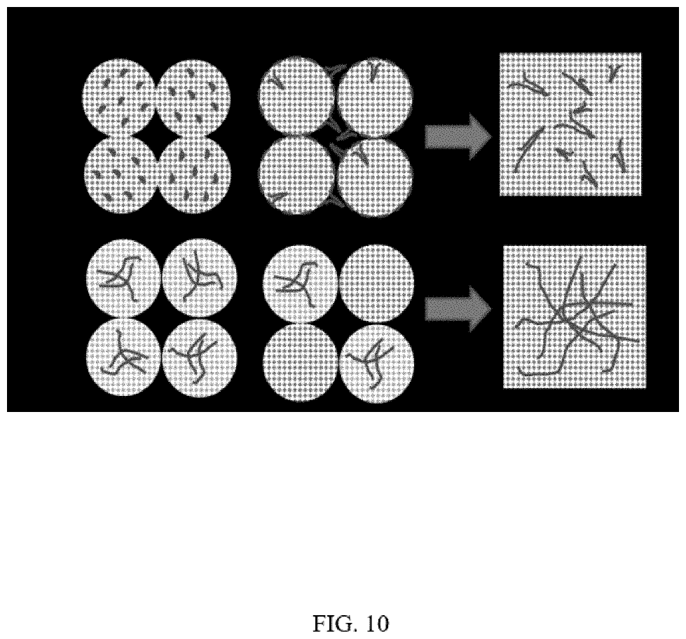

In certain embodiments, spheroids containing microvasculature capillary fragments can be directly assembled into larger constructs or intermixed at regular intervals with tissue-specific spheroids. FIG. 9 illustrates an embodiment depicting this concept. An array of tissue-specific spheroids and pre-formed MV-containing spheroids is depicted with each spheroid type arranged at a set distance from the other. Such an array can be used to seed microvascular networks within a biofabricated tissue construct. In certain embodiments, the spheroids fuse into larger structures, including, but not limited to, cardiac patches, vascular tubes, trachea, and urethra. In certain embodiments, endothelial cells (EC) are further added separate to or incorporated within each spheroid. In some embodiments, the endothelial cells survive and reorganize after an extended in vitro post-printing maturation, and/or after in vivo implantation. FIG. 10 depicts another embodiment of the disclosed method of using pre-formed MV-containing cell spheroids to seed microvascular networks within a biofabricated tissue construct.

### Experimental and Therapeutic Uses of Spheroids and Engineered Tissue Constructs

In certain embodiments, pre-formed MV-containing spheroids or engineered tissues containing pre-formed MV-containing spheroids can be used in vitro or in vivo as a minimalistic, discrete angiogenic environment, for mechanism discovery, drug discovery and substance testing.

In certain embodiments, pre-formed MV-containing spheroids or engineered tissues containing pre-formed MV-containing spheroids can be used in vitro or in vivo mixed with hydrogels or with other cells or other types of spheroids to test the efficacy of one or more drug substances on the spheroids themselves, or on another tissue type that is capable of subsisting due to the presence of the MV-containing spheroids.

In certain embodiments, pre-formed MV-containing spheroids containing only SMFCs or engineered tissues containing pre-formed MV-containing spheroids containing only SMFCs can be used as a cellular system for the secretion of paracrine factors that stimulate and/or support micro-vascular development in vivo.

In certain embodiments, pre-formed MV-containing spheroids or engineered tissues containing pre-formed MV-containing spheroids can be used to treat patients suffering from diseases, disorders, conditions, pathophysiological conditions and/or symptoms for which tissue replacement and/or cellular therapies are known to provide or are later found to provide therapeutic benefit.

In certain embodiments, cells used to form the MV-containing spheroids or engineered tissues containing pre-formed MV-containing spheroids are autologous, i.e., derived from the patient's own tissue.

In certain embodiments, cells used to form the MV-containing spheroids or engineered tissues containing pre-formed MV-containing spheroids are allogeneic, i.e., derived from a source other than the patient's own tissue.

In some embodiments, the engineered tissue containing pre-formed MV-containing spheroids as disclosed herein can be, for example, cardiac patches, vascular tubes, trachea, and urethra.

In certain embodiments, pre-formed MV-containing spheroids or engineered tissue containing pre-formed MV-containing spheroids can be used for the treatment or prophylaxis of angiogenesis-dependent or circulation-dependent diseases.

As used herein, the terms “treat,” “treatment,” or “treating,” refer to therapeutic treatments, wherein the object is to reverse, alleviate, ameliorate, inhibit, slow down or stop the progression or severity of a condition associated with a perfusion disorder or disease, e.g. an ischemia-reperfusion (FR) injury. The term “treating” includes reducing or alleviating at least one adverse effect or symptom of a condition, disease or disorder associated with a perfusion disorder. Treatment is generally “effective” if one or more symptoms or clinical markers are reduced. Alternatively, treatment is “effective” if the progression of a perfusion disorder is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

“Prophylaxis” or “prophylactic” or “preventative” therapy as used herein includes preventing the condition from occurring or ameliorating the subsequent progression of the condition in a subject that may be predisposed to the condition but has not yet been diagnosed as having it.

The term “allogeneic,” as used herein, refers to vascularized tissue constructs comprising cells of the same species as the transplant recipient but wherein the cells differ genetically. The term “autologous,” as used herein, refers to vascularized tissue constructs comprising cells derived from the same subject.

The term “engraft” as used herein refers to the process of incorporating a vascularized tissue construct into a tissue of interest in vivo through contact with existing cells of the tissue.

As used herein, the term “administering,” refers to the placement of a vascularized tissue construct as disclosed herein into a subject by a method or route which results in at least partial delivery of the composition at a desired site. In certain embodiments, the disclosed compositions can be administered to an organ or tissue ex vivo followed by their transplantation into the subject.

As used herein, angiogenesis-dependent or circulation-dependent diseases include, but are not limited to, ocular neovascular disease, retrolental fibroplasia, atherosclerosis, psoriasis, Crohn's diseases, neovascular glaucoma, retinopathy of prematurity, corneal graft rejection, retrolental fibroplasias, rubeosis, retinal neovascularization due to intervention, ocular tumors and trachoma, diabetic retinopathy, macular degeneration, neoplastic diseases, tumors of the bladder, brain, breast, cervix, colon, rectum, kidney, lung, ovary, pancreas, prostate, stomach and uterus, tumor metastasis, benign tumors, including but not limited to hemangiomas, acoustic neuromas, neurofibromas, trachomas, and pyrogenic granulomas, hypertrophy, including but not limited to cardiac hypertophy, immune and non-immune inflammation, chronic articular rheumatism and psoriasis, restenosis, capillary proliferation in atherosclerotic plaques and osteoporosis, and cancer associated disorders, including but not limited to solid tumors, solid tumor metastases, angiofibromas, retrolental fibroplasia, hemangiomas, Kaposi sarcoma, lymphoid malignancies, including but not limited to chronic and acute lymphoid leukemias, and lymphomas.

In certain embodiments, pre-formed MV-containing spheroids or engineered tissues containing pre-formed MV-containing spheroids are used in the treatment or prophylaxis of a perfusion disorder. Perfusion is the process by which a fluid passes through the circulatory system or lymphatic system of an organ, tissue, or extremity, e.g. the delivery of blood to a capillary bed in a tissue.

As used herein, a “perfusion disorder” or “perfusion disease” is any pathological process that deprives a subject's tissue, organ or extremity of oxygenated blood. A perfusion disorder can be caused by physical trauma or as a consequence of systemic or vascular disease that reduces arterial flow to an organ, tissue and/or extremity. Physical trauma can include, for example, a chronic obstructive process, or injury resulting from a physical insult such as frostbite or radiation.

Examples of a perfusion disorder include, but are not limited to, vascular disease, atherosclerosis, peripheral artery disease (PAD), critical limb ischemia (CLI), restenosis or vulnerable plaque.

As used herein, a “vascular disease” refers to a perfusion disorder of the blood vessels, primarily arteries and veins, which transport blood to and from the heart, lungs, brain and peripheral organs such as, without limitation, the arms, legs, kidneys and liver. In particular, “vascular disease” refers to the coronary arterial and venous systems, the carotid arterial and venous systems, the aortic arterial and venous systems and the peripheral arterial and venous systems. The disease that may be treated is any that is amenable to treatment with the compositions disclosed herein, either as the sole treatment protocol or as an adjunct to other procedures such as surgical intervention.

“Atherosclerosis” refers to the depositing of fatty substances, cholesterol, cellular waste products, calcium and fibrin on the inner lining or intima of an artery. Smooth muscle cell proliferation and lipid accumulation accompany the deposition process. In addition, inflammatory substances that tend to migrate to atherosclerotic regions of an artery are thought to exacerbate the condition. The result of the accumulation of substances on the intima is the formation of fibrous (atheromatous) plaques that can occlude the lumen of the artery, a process called stenosis. When the stenosis becomes severe enough, the blood supply to the organ supplied by the particular artery is depleted resulting in a stroke, if the afflicted artery is a carotid artery, or a heart attack if the artery is coronary, or loss of organ or limb function if the artery is peripheral.

Peripheral vascular diseases are generally caused by structural changes in blood vessels caused by such conditions as inflammation and tissue damage. A subset of peripheral vascular disease is peripheral artery disease (PAD). PAD is a condition that is similar to carotid and coronary artery disease in that it is caused by the buildup of fatty deposits on the lining or intima of the artery walls. Just as blockage of the carotid artery restricts blood flow to the brain and blockage of the coronary artery restricts blood flow to the heart, blockage of the peripheral arteries can lead to restricted blood flow to the kidneys, stomach, arms, legs and feet. Peripheral vascular disease includes arterial and venous diseases of the renal, iliac, femoral, popliteal, tibial and other vascular regions. In particular, a peripheral vascular disease can refer to a vascular disease of the superficial femoral artery.

“Critical limb ischemia” (CLI) is an advanced stage of peripheral artery disease (PAD). It is defined as a triad of ischemic rest pain, arterial insufficiency ulcers, and gangrene. The latter two conditions are jointly referred to as tissue loss, reflecting the development of surface damage to the limb tissue due to the most severe stage of ischemia. Over 500,000 patients in the U.S. each year are diagnosed with critical limb ischemia (CLI). Half the patients die from a cardiovascular cause within 5 years, a rate that is 5 times higher than a matched population without CLI (Varuet al. (2010) Journal of Vascular Surgery 51(1): 230-41; Rundback et al. Ann Vasc Surg. (2017) 38:191-205).

“Restenosis” refers to the re-narrowing of an artery at or near the site where angioplasty or another surgical procedure was previously performed to remove a stenosis. It is generally due to smooth muscle cell proliferation and, at times, is accompanied by thrombosis.

“Vulnerable plaque” refers to an atheromatous plaque that has the potential of causing a thrombotic event and is usually characterized by a thin fibrous cap separating a lipid filled atheroma from the lumen of an artery. The thinness of the cap renders the plaque susceptible to rupture. When the plaque ruptures, the inner core of usually lipid-rich plaque is exposed to blood. This releases tissue factor and lipid components with the potential of causing a potentially fatal thrombotic event through adhesion and activation of platelets and plasma proteins to components of the exposed plaque.

### EXAMPLES

The materials, methods, and embodiments described herein are further defined in the following Examples. Certain embodiments are defined in the Examples herein. It should be understood that these Examples, while indicating certain embodiments, are given by way of illustration only. From the disclosure herein and these Examples, one skilled in the art can ascertain the essential characteristics of this invention, and without departing from the spirit and scope thereof, can make various changes and modifications of the invention to adapt it to various usages and conditions.

The practice of the invention employs, unless otherwise indicated, conventional molecular and cellular biological as well as immunological techniques within the skill of those in the art. Such techniques are well known to the skilled worker and are explained fully in the literature. See, e.g., Bailey, J. E. and Ollis, D. F., Biochemical Engineering Fundamentals, McGraw-Hill Book Company, N Y, 1986; Current Protocols in Immunology, John Wiley & Sons, Inc., NY, N.Y. (1991-2015), including all supplements; Current Protocols in Molecular Biology, John Wiley & Sons, Inc., NY, N.Y. (1987-2015), including all supplements; Sambrook, et al., Molecular Cloning: A Laboratory Manual, 2nd Edition, Cold Spring Harbor, N.Y. (1989); and Harlow and Lane, Antibodies, a Laboratory Manual, Cold Spring Harbor, N.Y. (1989).

### Example 1: Formation of Compact ECFCs-Containing Spheroids

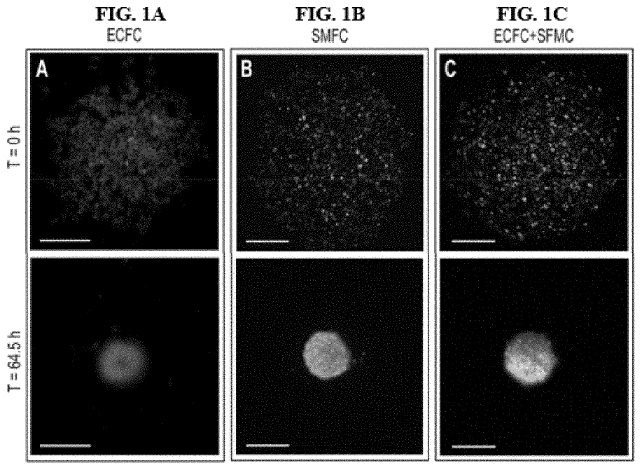

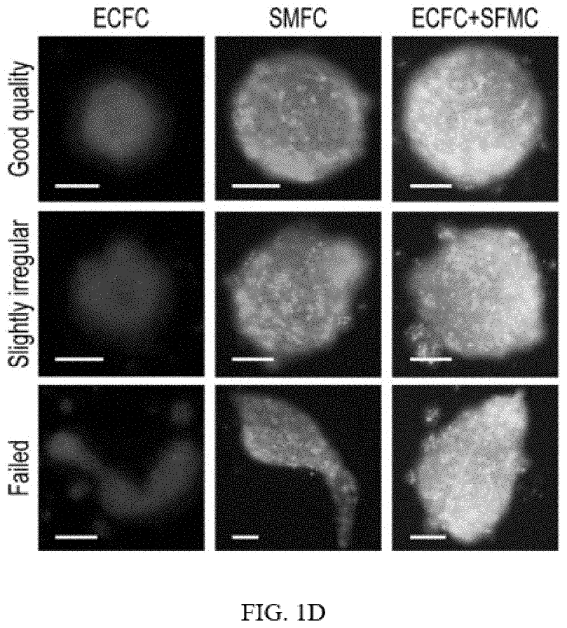

Culturing/seeding of NRP⁺CD31⁺ ECFCs (iPSC-derived vascular cells) in ultralow-adhesive, U-shaped well culture plates produced cell lawns with a scattered pattern (FIG. 1A top, images taken on the IncuCyte automatic microscope). These colonies eventually form spheroids, many of which are not uniformly compact and/or imperfectly round (FIG. 1A, bottom), indicating a reduced intercellular adhesiveness between ECFCs. FIG. 1A depicts the tdTomato labeled iPSC-derived ECFCs as diffuse colonies (top), that formed spheroids having an irregular shape (bottom).

eGFP⁺/SMFCs (iPSC-derived smooth muscle cells) formed round discs (FIG. 1B, top), that condensed into almost spherical cell clusters (FIG. 1B, bottom). For this reason, the two cell populations were combined, to see if SMFCs could provide the ECFCs with added support needed to the production of sturdier spheroids. Indeed, the mixed tdTomato⁺-ECFC/eGFP⁺ SMFC colonies (FIG. 1C top, mixed at a ratio of 1:5 iPSC-derived ECFCs to SMFCs) displayed the same kinetics, and formed the same compact shape (FIG. 1C, bottom) as eGFP/SMFCs. FIGS. 1A-1D depict representative images selected from 80-90 spheroids/condition. The frequency in the formation of imperfectly formed spheroids correlated with the type of cells incorporated into the spheroids. The highest number of imperfect spheroids were observed with ECFs, followed by SMFCs whereas the spheroids comprising ECFCs and SMFCs were the most well-rounded and compact. FIG. 1D depicts images of types of spheroids found at the end of a four-day incubation. ECFC-only spheroids presented a high frequency of incomplete formation (˜30%) and of failure (˜30%). SMFC-only and ECFC/SMFC spheroids were almost always round and compact.

### Example 2: Spheroid Size Stabilization

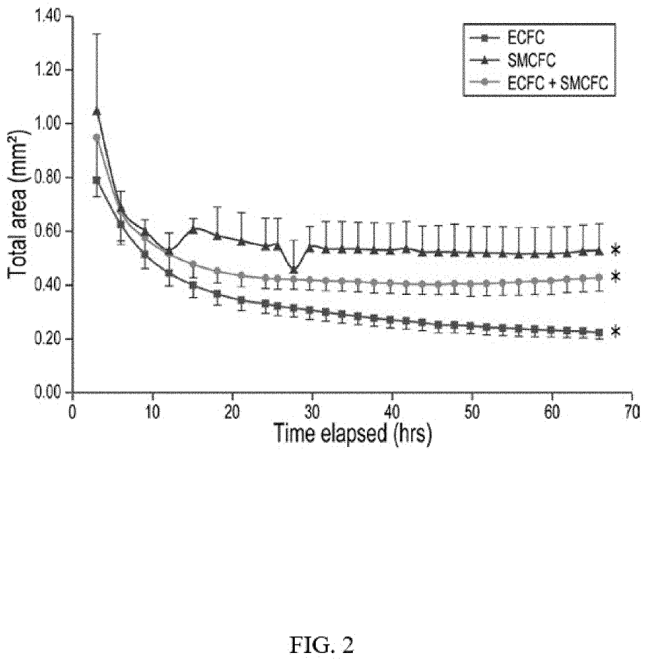

Putting the spheroids into use prematurely, i.e., without sufficient compaction, can lead to biofabricated tissue constructs having suboptimal properties. Thus, determining the size and cellular composition of spheroids ensures the selection of spheroids having the requisite stability both in terms of cell turnover, i.e. proliferation and death and integrity. To this end, the area of fluorescent images was measured in all three conditions using IncuCyte's software (FIG. 2). This analysis confirmed the visual observation that, although all spheroids were made from the same initial number of cells, ECFC spheroids were not only more irregular, but also smaller (see FIGS. 1A-1D). In addition, this quantitation demonstrated that after a rapid shrinking, the SMFC-containing spheroids size stabilizes after about 18 hours, while that of ECFC-only spheroids continues to decrease (FIG. 2). Surprisingly, the size of mixed spheroids remained larger than that of ECFC-only spheroids, and also stabilized, even showing a trend towards an increase (FIG. 2). FIG. 2 depicts a time course of spheroid size as determined from the fluorescence images (squares=ECFC only (n=57); triangles=SMFC (n=87); circles=mixed in the proportion 1:5 (ECFC/SMFC) (n=93)). Data are presented as mean±SD, p<0.001 for all comparisons, at the end of the incubation.

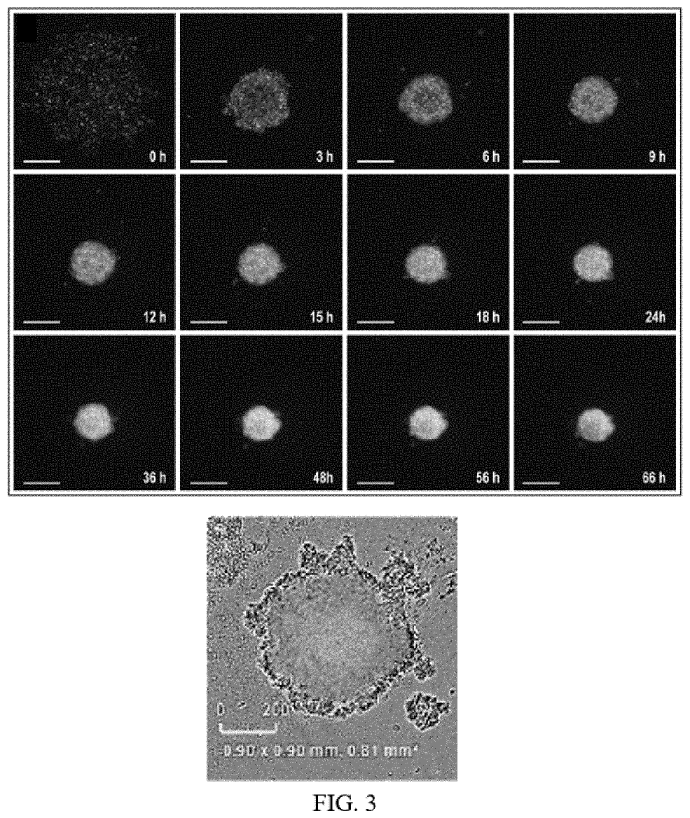

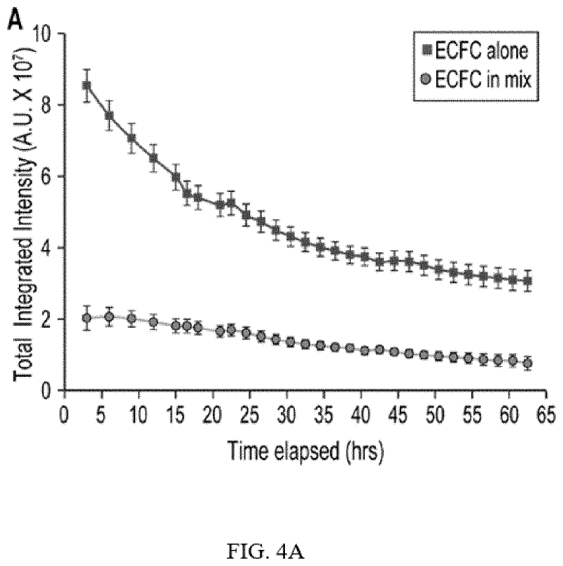

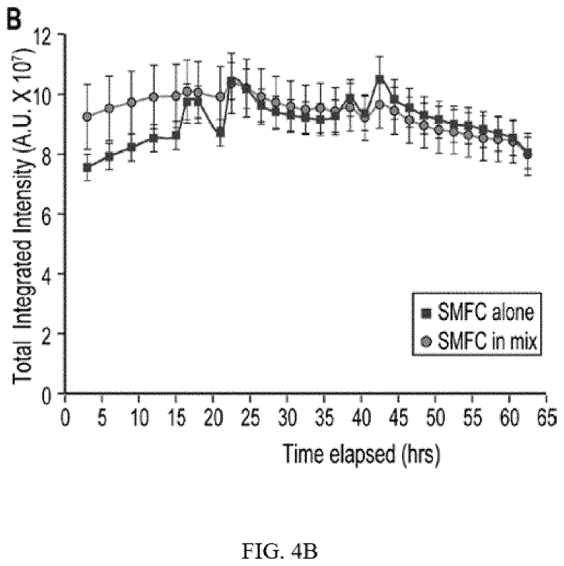

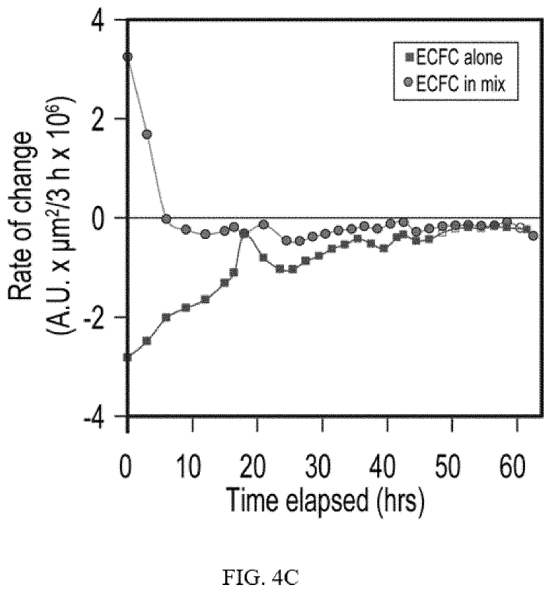

Time-lapse microscopy of mixed spheroids demonstrated that size stabilization coincides with a temporary stabilization of cellular composition after about 24 hours incubation (see FIG. 3; result corroborated by data of FIGS. 4A-4C). A relatively stable spheroid size was attained at ˜18 hours, but the cell proportions on the surface changed from predominantly ECFCs (red) at 9 hours, to mostly SMFCs (green) at 48 hours. Overall the apparent cellular composition changed, as indicated by observations at the spheroid's surface, which displayed an increase in the proportion of eGFP⁺/SMFCs (see FIG. 3; compare the early and late frames). This shift is indicative of ECFCs translocation from the surface toward the spheroid's core, or center, and/or reduction of their proportion, given that the core eventually becomes positive for caspase 3/7 activity (see FIG. 3, last frame).

### Example 3: Intra-Spheroid Cellular Kinetics

The light intensity of spheroids on the two fluorescence channels was quantified and assumed to be proportional to the number of respective emitting cells, as dependent on the time in culture. This allowed a comparison of behavior and the superficial (FIG. 3) vs. overall (FIGS. 4A-4C) cell proportions in spheroids. FIG. 4A depicts a time course of fluorescence of spheroids made from ECFCs only (squares, n=57), and of tdTomato fluorescence in mixed 1:5 ECFC/SMFC spheroids (circles, n=93). FIG. 4B. depicts the fluorescence of the spheroids prepared from eGFP⁺/NRP1⁻CD31⁺ cells (squares, n=87), and of the eGFP in mixed 1:5 ECFC/SMFC spheroids (circles, n=93). The plates were seeded with cells from the same batch and processed and imaged in parallel. Data represent mean integrated intensity/image ±SD.

As depicted in FIGS. 4A-4C, the fluorescence intensity produced by the spheroids made from ECFCs alone decreased steadily (FIG. 4A, squares), whereas in mixed spheroids the red fluorescence produced by the tdTomato positive ECFCs was more stable (FIG. 4A, circles). This difference in fluorescence change rates was better documented by these two curves' derivatives, which indicate the speed of change (FIG. 4C). At the same time, the fluorescence produced by the eGFP⁺ SMFCs either alone (FIG. 4B, squares) or in combination (FIG. 4B, circles) slightly increased and stabilized in the first two days, after which the fluorescence started to decline. These data indicate that ECFC-only clusters are structurally more unstable than SMFC-only clusters maintained in culture, but the latter can provide ECFCs with protection in mixed spheroids.

### Example 4: Cellular Relocation within Spheroids

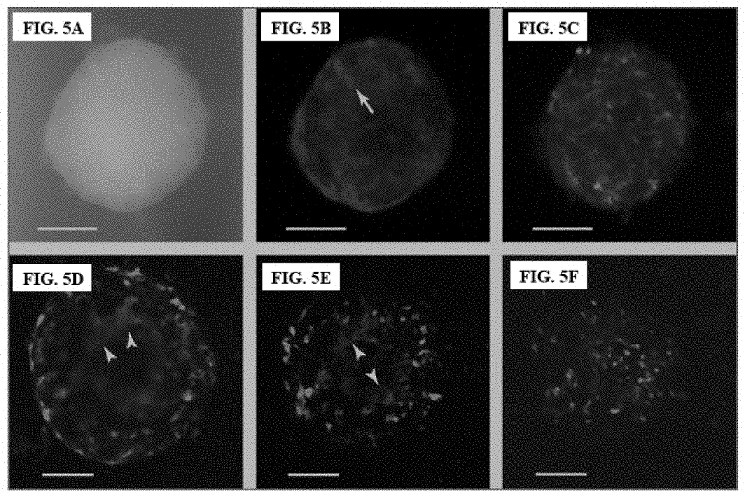

One of the conspicuous properties of cell-heterogeneous spheroids is cell sorting, namely separation of cell layers based on their affinity: those with increased adhesiveness compacting themselves more at the core. Because adult endothelial cells tend toremain at the surface of mixed cell spheroids, along with ECFCs' reduced intercellular adhesiveness as manifested in the early stages of aggregation (see FIG. 1A vs. 1B), it was anticipated that ECFCs would also prefer a superficial distribution. Instead, after initially displaying the expected surface localization, surprisingly the proportion of superficial ECFCs dramatically decreased, with the majority of cells eventually visible from the exterior being eGFP-positive (FIG. 3). This process was confirmed by imaging the spheroids at higher magnification (FIG. 5A-5F). FIG. 5 depicts the cell distribution in early vs. late spheroids, with FIG. 5A presenting phase contrast imaging of a mixed one-day old spheroid, FIG. 5B presenting radial positioning of a tdTomato-positive iPSC-ECFC cell cord within this early spheroid (arrow), FIG. 5C presenting corresponding eGFP-expressing SMFCs in the same spheroid, and FIGS. 5D-5F presenting successive confocal sections through a one-week old spheroid (bars: FIG. 5A-5C, 200 μm; FIG. 5D-5F, 100 μm).

Notably, FIG. 5B indicates superficial ECFC accumulations and also cell cords, as part of the cell dynamics in these live structures and extensive cell sorting (layered separation), and the organization of the ECFCs in a fashion suggestive of cell cord formation, with sprouting viewed longitudinally (FIG. 5D, arrowheads), or transversally (FIG. 5E, arrowheads). Optical sectioning by confocal microscopy of older spheroids revealed an almost complete cell sorting, with cortical distribution of SMFCs, and ECFCs concentrated at the core (see FIGS. 5D-5F).

Since the core of spheroids maintained long-term in culture becomes positive for caspase 3/7 activity (FIG. 3, last frame), it may be assumed that during this translocation the ECFCs are under increasing ischemic stress, and thus become more vulnerable to cell loss, as actually observed by the kinetic fluorescence analysis (FIG. 3). However, this supposition is contradicted by the structured organization of ECFCs at the core of these spheroids, indicative of not only cell cord formation (FIG. 5B), but also of possible sprouting (FIG. 5D).

### Example 5: SMFCs-Containing Spheroids can be Assembled in Larger Structures by Scaffold-Free Biofabrication

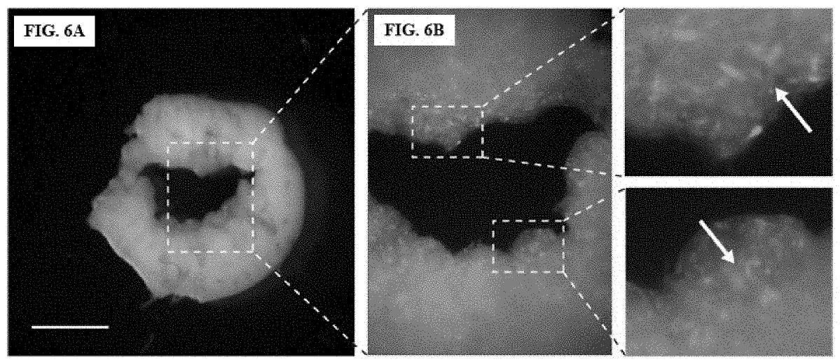

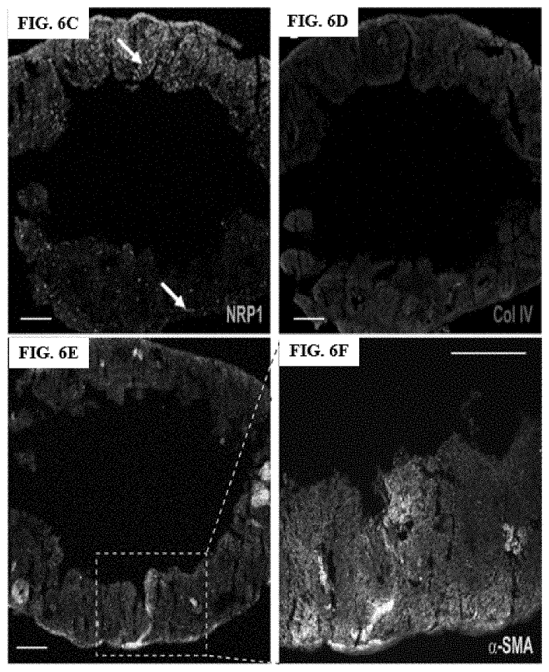

Next, it was verified whether the iPSC-derived spheroids can undergo fusion either independently, or after being laced together in larger structures. To this end, separately-formed spheroids were re-incubated in the same wells, and the results indicated that their fusion took place, progressing with a pace dependent on the cluster size (see FIGS. 6A-6B vs. FIGS. 6C-6D).

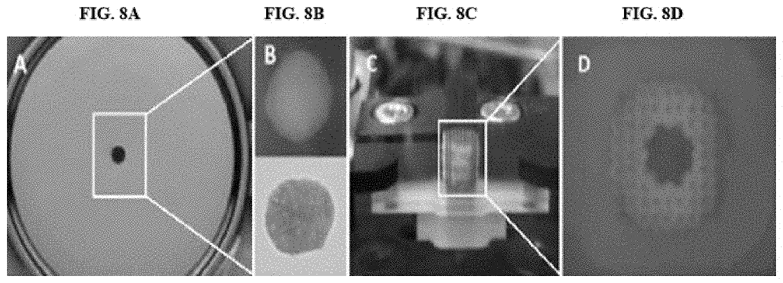

To keep the spheroids in more controlled contact, for longer intervals, and in larger assemblies, the Regenova™ Bio 3D-Printing robot was used. This instrument ‘skewers’, one-by-one, pre-formed half-millimeter cell spheroids in stainless steel microneedles (‘Kenzans’). In this method, the spheroids are skewered in contact with each other and maintained in place for post-printing maturation, when they may fuse into tissue-like structures by cell translocation and further matrix secretion (FIGS. 8A-8D).

FIGS. 6A-6F depict the characterization of a construct prepared on the Regenova™ bioprinter from iPSC-derived SMFCs. FIG. 6A depicts a micro-photograph of the construct after one week of post-printing maturation. FIG. 6B depicts whole-mount fluorescence microscopy, showing the eGFP-expressing cells (arrows, in the inserts), embedded in abundant, newly-secreted extracellular matrix (ECM) (pale fluorescence, auto-fluorescence). FIG. 6C depicts a confocal microscopy image of the construct, identifying the eGFP-SMFCs (arrows). FIG. 6D depicts the same section as FIG. 6C with immunostaining for collagen type IV (red). FIG. 6E demonstrates that CD31⁺NRP1⁻ cells differentiate into alpha-smooth muscle actin in positive (yellow) and negative (fibroblastic) cells, the former being more concentrated on the surfaces of the construct. FIG. 6F is a higher magnification image of FIG. 6E. In FIGS. 6C-6F, blue represents DAPI staining. Magnification bar (FIG. 6A): 2 mm.

In accord with the observations in individual spheroids, the ECFCs retained the ability to fuse and form ECFC cell cords even after their assembling on the Kenzan (FIG. 6E), unlike the co-embedded SMFCs (FIG. 6F).

### Example 6: Bioprinted Cells Show Signs of Vascular Differentiation

Previous attempts to permanently lace by this method cell spheroids made only from the ECFCs have failed, consistent with their poorer aggregation capacity (FIGS. 1A-1D). In contrast, those made from CD31⁺NRP1⁻ cells were readily Kenzan-printable (FIGS. 6A-6F). Their consistency was strong enough to resist the gentle aspiration by the nozzle connected to the robotic arm of the bioprinter, and the piercing by the sharp microneedles. The bioprinted construct was maintained in a micro-needle array with a hollow space, which allows the tubular construct's continuous perfusion with fresh medium. Then this tissue was removed, fixed, and either imaged by light transmission microscopy (FIG. 6A), or by fluorescence microscopy (FIG. 6B), or immuno-stained after embedment in paraffin and sectioning. Based on the distribution of DAPI-positive nuclei (FIGS. 6C-6F) this construct represents a densely-populated cellular structure, with many of the cells still expressing the eGFP marker in various degrees (FIG. 6C). However, their distribution was rather sparse (FIG. 6B), with space likely occupied by other cells, or by ECM rich in collagen type IV (FIG. 6D). Surprisingly, this construct also presented a layered distribution of smooth-muscle actin-expressing cells (FIG. 6E). The cells positive for this actin isoform were distributed either closer to the interior, or to the exterior of the tube, but was largely missing in the middle section (FIG. 6F).

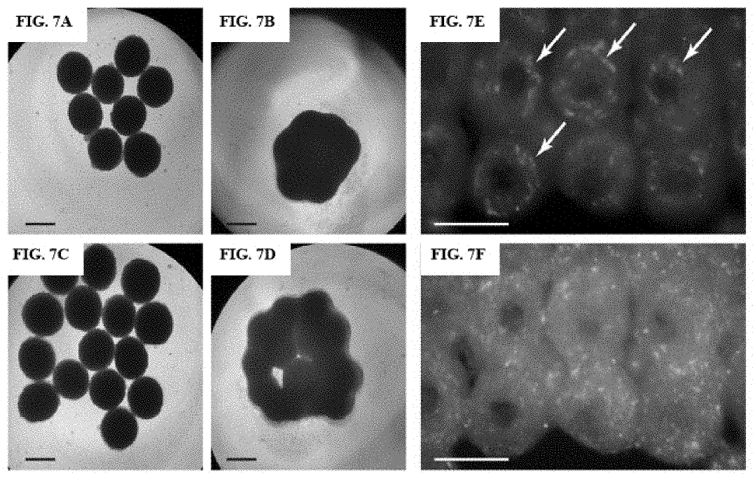

FIG. 7A depicts the characterization of in-well fusion and internal organization of iPSC-derived spheroids in eight (FIGS. 7A-7B) and 16 (FIGS. 7C-7D) mixed 1:10 ECFC/SMFC, 24 hours-old spheroids. FIGS. 7A & 7C depict images taken at time 0 while FIGS. 7B & 7D depict images taken at 48 hours. FIGS. 7E-7F depict Kenzan assembling of mixed spheroids. FIG. 7E depicts ECFCs (red): note the extended, cord-like circular organization of ECFCs in these bioprinted spheroids immediately after removal from the micro-needle array. FIG. 7F depicts corresponding SMFCs (green).

### Materials and Methods

### Cell Isolation and Labeling.

Human iPSC and ECFCs were obtained and expanded by previously published methods (Prasain, N. et al., Differentiation of Human Pluripotent Stem Cells Similar to Cord-Blood Endothelial Colony-Forming Cells, Nat Biotechnol, 32(11):1151-1157 (2014)). hESCs and hiPSCs were maintained in mTeSR1 complete medium (Stem Cell Technologies) on Matrigel in 10 cm² tissue culture dishes at 37° C. and 5% CO2. After the plating of cells, medium was changed on days 2, 3 and 4. Cells were passaged on day 5. Medium was aspirated and 4-5 ml of dispase (2 mg/ml, Gibco) containing medium was added to the plate and incubated at 37° C. for 3-5 min or until the edges of the colonies had lifted from the plate. Dispase containing medium was aspirated from the plate and cells were gently washed with DMEM-F12 (Gibco) three times to remove any residual amount of enzyme. Fresh medium was then used to collect colonies from the plate using a forceful wash and scraping with a 5-ml disposable pipette taking care to avoid bubbles. Collected colonies were then centrifuged at 300 g for 5 min. The supernatant was aspirated and pellet was resuspended in mTeSR1 complete medium. Prior to passaging, 10 cm² tissue culture dishes were coated with Matrigel for 30 min. Unattached Matrigel was removed from the tissue culture dishes and 7 ml of mTeSR1 complete medium was added to dishes. Colonies evenly distributed in mTeSR1 medium were added to each plate. Cells were then spread out within the dish using multiple side-to-side shaking motions while avoiding any swirling. Cultures were checked for growth quality and morphology, and by performing teratoma formation assay as previously described CD31⁺NRP1⁻ (SMFC progenitor) cells were obtained in the same selection as the CD31⁺NRP1⁺ (ECFC progenitor) cells. These cells were transduced to express eGFP and tdTomato, respectively, using a lentiviral vector. Both cell types were routinely maintained and passed in complete EGM-2 medium (Lonza).

### Spheroid Formation.

Labeled cells were seeded in EGM-2 medium in ultra-low adhesive U-bottomed 96 well plates (Sumitomo Bakelite, Tokyo, Japan). After testing various cell concentrations, it was determined that 2.5×10⁴ cells/well produce within 24 hours spheroids of about 0.5 mm diameter, as required for Regenova™ bioprinting. This initial cell number was maintained for preparation of spheroids throughout the experiments. To test their propensity for fusion, after separate formation the spheroids were placed in the same wells for up to 72 hours.

### Spheroid Imaging and Analysis.

After seeding, the plates were monitored every 4 hours for 72 hours in the automated microscope IncuCyte (Essen Bioscience). In select cases, after 52 hours an apoptosis reagent caspase 3/7 substrate was added, according to provider's instructions (Essen Biosciences). Alternatively, at the end of the incubation the spheroids were imaged in combined transmission/fluorescence microscopy (Leica DMIL), or in a confocal Olympus FV1000 MPE microscope.

### Kenzan Bioprinting.

Spheroids prepared for 24 hours were used for bioprinting. The Kenzan method performed by the Regenova™ bioprinter consists of gentle aspiration of individual spheroids from their formation wells, and placement in contact with surrounding spheroids by implantation on stainless steel micro-needles (‘kenzans’) of 170 microns in diameter. These microneedles were arranged in a Cartesian pattern with a hollow central space, which allows the perfusion of fresh EGM-2 medium. The construct was maintained for a week or more on the needle array to allow spheroids to fuse and produce extracellular matrix, in a perfusion bioreactor operated at a speed of 4 ml/min.

### Immunostaining

At the end of incubation, the construct was removed and either directly imaged in fluorescence microscopy, or fixed in 4% paraformaldehyde, embedded in OCT, and then sectioned on a Leica microtome. Sections were stained with antibodies to alpha smooth muscle actin or collagen type IV, and counter-stained with DAPI for nuclei localization.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology.

1-54. (canceled)

55. A population of spheroids, wherein each spheroid comprises endothelial progenitor cells (EPCs) and CD31⁺NRP1⁻ smooth muscle forming cells (SMFCs).

56. The population of spheroids of claim 55, wherein the EPCs comprise CD31⁺NRP1⁺ endothelial colony forming cells (ECFCs).

57. The population of spheroids of claim 55, wherein the EPCs and SMFCs are derived from a pluripotent stem cell population.

58. The population of spheroids of claim 57, wherein the pluripotent stem cell population comprises induced pluripotent stem cells (iPSCs).

59. The population of spheroids of claim 55, wherein the EPCs are derived from peripheral blood or cord blood.

60. The population of spheroids of claim 55, wherein each spheroid is between about 200 microns and about 600 microns in diameter and comprises about 0.5% to about 20% EPCs and about 80% to about 99.5% SMFCs.

61. The population of spheroids of claim 55, wherein each spheroid comprises about 20% EPCs and about 80% SMFCs.

62. The population of spheroids of claim 55, wherein one or more capillary fragments form at the center of each spheroid.

63. An engineered tissue comprising the population of spheroids of claim 55.

64. A method of producing a vascularized tissue construct, comprising:
co-culturing endothelial colony progenitor cells (EPCs) and smooth muscle forming cells (SMFCs) to form a population of spheroids; and
bioprinting the population of spheroids into a tissue construct.

65. The method of claim 64, wherein the EPCs and SMFCs are co-cultured for about 18 to 48 hours on an ultra-low attachment plate.

66. The method of claim 65, wherein the tissue construct is scaffold-free.

67. The method of claim 66, wherein the bioprinting comprises skewering the population of spheroids onto microneedles in a desired configuration.

68. The method of claim 64, wherein the bioprinting comprises Kenzan bioprinting.

69. The method of claim 64, wherein cells within each spheroid translocate during or after bioprinting to form a plurality of microvascular structures within the tissue construct.

70. The method of claim 64, wherein one or more spheroids in the tissue construct contact each other.

71. A method of seeding microvascular networks within a tissue construct, comprising bioprinting the tissue construct with a first population of tissue-specific spheroids.

72. The method of claim 71, further comprising combining the first population of tissue-specific spheroids with adult capillary fragments embedded with mesenchymal stem cells.

73. The method of claim 71, wherein the tissue construct comprises a first spheroid population comprising EPCs and CD31+NRP1− SMFCs interspersed with a second population of tissue-specific spheroids.

74. The method of claim 71, wherein the tissue construct is a cardiac patch, a vascular tube, a trachea, or a urethra.


SEQUENCE LISTING (4 sequences)

SEQ ID NO: 1 - DNA, 2217 bp, Homo sapiens
atgcagccgaggtgggcccaaggggccacgatgtggcttggagtcctgctgacccttctgctctgttcaagccttgagggtcaagaaaactctttcacaatcaacagtgttgacatgaagagcctgccggactggacggtgcaaaatgggaagaacctgaccctgcagtgcttcgcggatgtcagcaccacctctcacgtcaagcctcagcaccagatgctgttctataaggatgacgtgctgttttacaacatctcctccatgaagagcacagagagttattttattcctgaagtccggatctatgactcagggacatataaatgtactgtgattgtgaacaacaaagagaaaaccactgcagagtaccaggtgttggtggaaggagtgcccagtcccagggtgacactggacaagaaagaggccatccaaggtgggatcgtgagggtcaactgttctgtcccagaggaaaaggccccaatacacttcacaattgaaaaacttgaactaaatgaaaaaatggtcaagctgaaaagagagaagaattctcgagaccagaattttgtgatactggaattccccgttgaggaacaggaccgcgttttatccttccgatgtcaagctaggatcatttctgggatccatatgcagacctcagaatctaccaagagtgaactggtcaccgtgacggaatccttctctacacccaagttccacatcagccccaccggaatgatcatggaaggagctcagctccacattaagtgcaccattcaagtgactcacctggcccaggagtttccagaaatcataattcagaaggacaaggcgattgtggcccacaacagacatggcaacaaggctgtgtactcagtcatggccatggtggagcacagtggcaactacacgtgcaaagtggagtccagccgcatatccaaggtcagcagcatcgtggtcaacataacagaactattttccaagcccgaactggaatcttccttcacacatctggaccaaggtgaaagactgaacctgtcctgctccatcccaggagcacctccagccaacttcaccatccagaaggaagatacgattgtgtcacagactcaagatttcaccaagatagcctcaaagtcggacagtgggacgtatatctgcactgcaggtattgacaaagtggtcaagaaaagcaacacagtccagatagtcgtatgtgaaatgctctcccagcccaggatttcttatgatgcccagtttgaggtcataaaaggacagaccatcgaagtccgttgcgaatcgatcagtggaactttgcctatttcttaccaacttttaaaaacaagtaaagttttggagaatagtaccaagaactcaaatgatcctgcggtattcaaagacaaccccactgaagacgtcgaataccagtgtgttgcagataattgccattcccatgccaaaatgttaagtgaggttctgagggtgaaggtgatagccccggtggatgaggtccagatttctatcctgtcaagtaaggtggtggagtctggagaggacattgtgctgcaatgtgctgtgaatgaaggatctggtcccatcacctataagttttacagagaaaaagagggcaaacccttctatcaaatgacctcaaatgccacccaggcattttggaccaagcagaaggctagcaaggaacaggagggagagtattactgcacagccttcaacagagccaaccacgcctccagtgtccccagaagcaaaatactgacagtcagagtcattcttgccccatggaagaaaggacttattgcagtggttatcatcggagtgatcattgctctcttgatcattgcggccaaatgttattttctgaggaaagccaaggccaagcagatgccagtggaaatgtccaggccagcagtaccacttctgaactccaacaacgagaaaatgtcagatcccaatatggaagctaacagtcattacggtcacaatgacgatgtcagaaaccatgcaatgaaaccaataaatgataataaagagcctctgaactcagacgtgcagtacacggaagttcaagtgtcctcagctgagtctcacaaagatctaggaaagaaggacacagagacagtgtacagtgaagtccggaaagctgtccctgatgccgtggaaagcagatactctagaacggaaggctcccttgatggaacttag

SEQ ID NO: 2 - protein, 738 aa, Homo sapiens
MQPRWAQGATMWLGVLLTLLLCSSLEGQENSFTINSVDMKSLPDWTVQNGKNLTLQCFADVSTTSHVKPQHQMLFYKDDVLFYNISSMKSTESYFIPEVRIYDSGTYKCTVIVNNKEKTTAEYQVLVEGVPSPRVTLDKKEAIQGGIVRVNCSVPEEKAPIHFTIEKLELNEKMVKLKREKNSRDQNFVILEFPVEEQDRVLSFRCQARIISGIHMQTSESTKSELVTVTESFSTPKFHISPTGMIMEGAQLHIKCTIQVTHLAQEFPEIIIQKDKAIVAHNRHGNKAVYSVMAMVEHSGNYTCKVESSRISKVSSIVVNITELFSKPELESSFTHLDQGERLNLSCSIPGAPPANFTIQKEDTIVSQTQDFTKIASKSDSGTYICTAGIDKVVKKSNTVQIVVCEMLSQPRISYDAQFEVIKGQTIEVRCESISGTLPISYQLLKTSKVLENSTKNSNDPAVFKDNPTEDVEYQCVADNCHSHAKMLSEVLRVKVIAPVDEVQISILSSKVVESGEDIVLQCAVNEGSGPITYKFYREKEGKPFYQMTSNATQAFWTKQKASKEQEGEYYCTAFNRANHASSVPRSKILTVRVILAPWKKGLIAVVIIGVIIALLIIAAKCYFLRKAKAKQMPVEMSRPAVPLLNSNNEKMSDPNMEANSHYGHNDDVRNHAMKPINDNKEPLNSDVQYTEVQVSSAESHKDLGKKDTETVYSEVRKAVPDAVESRYSRTEGSLDGT

SEQ ID NO: 3 - DNA, 2772 bp, Homo sapiens
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaaattttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccccacagatgttgtggttgcagtattccccaaaccactgataactcgatttgtccgaatcaagcctgcaacttgggaaactggcatatctatgagatttgaagtatacggttgcaagataacagattatccttgctctggaatgttgggtatggtgtctggacttatttctgactcccagatcacatcatccaaccaaggggacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaccccatcctcatcaccatcatagccatgagtgccctgggggtcctcctgggggctgtctgtggggtcgtgctgtactgtgcctgttggcataatgggatgtcagaaagaaacttgtctgccctggagaactataactttgaacttgtggatggtgtgaagttgaaaaaagacaaactgaatacacagagtacttattcggaggcatga

SEQ ID NO: 4 - protein, 923 aa, Homo sapiens
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDPILITIIAMSALGVLLGAVCGVVLYCACWHNGMSERNLSALENYNFELVDGVKLKKDKLNTQSTYSEA